(19) United States
(12) Patent Application Publication
(10) Pub. No.: US 2003/0186994 A1
(43) Pub. Date: Oct. 2, 2003

(54) Combination of statins and sorbitol dehydrogenase inhibitors

(76) Inventor: Banavara L. Mylari, Waterford, CT (US)

Correspondence Address: Gregg C. Benson, Pfizer Inc., Patent Department, MS 4159, Eastern Point Road, Groton, CT, 06340, US

(21) Appl. No.: 09/974,414

(22) Filed: Oct. 9, 2001

Related U.S. Application Data
(60) Provisional application No. 60/241,339, filed on Oct. 18, 2000.

(51) Int. Cl. A61K 31/505; A61K 31/401; A61K 31/366; A61K 31/225

(52) U.S. Cl. 514/256; 514/423; 514/460; 514/548

(57) ABSTRACT

This invention relates to pharmaceutical compositions comprising combinations of a statin, a prodrug thereof or a pharmaceutically acceptable salt of said statin or said prodrug and a sorbitol dehydrogenase inhibitor, a prodrug thereof or a pharmaceutically acceptable salt of said sorbitol dehydrogenase inhibitor or said prodrug, kits containing such combinations and methods of using such combinations to treat mammals, including humans, suffering from atherosclerosis and/or diabetic complications such as diabetic neuropathy, diabetic nephropathy, diabetic cardiomyopathy, diabetic retinopathy, diabetic microangiopathy, diabetic macroangiopathy, cataracts or foot ulcers.

This invention relates to pharmaceutical combinations of a statin, a prodrug thereof or a pharmaceutically acceptable salt of said statin or said prodrug and a sorbitol dehydrogenase inhibitor, a prodrug thereof or a pharmaceutically acceptable salt of said sorbitol dehydrogenase inhibitor or said prodrug, kits containing such combinations and methods of using such combinations to treat mammals, including humans, suffering from atherosclerosis and/or diabetic complications such as, inter alia, diabetic neuropathy, diabetic nephropathy, diabetic cardiomyopathy, diabetic retinopathy, diabetic microangiopathy, diabetic macroangiopathy, cataracts or foot ulcers. This invention also relates to additive and synergistic combinations of a statin, a prodrug thereof or a pharmaceutically acceptable salt of said statin or said prodrug and a sorbitol dehydrogenase inhibitor, a prodrug thereof or a pharmaceutically acceptable salt of said sorbitol dehydrogenase inhibitor or said prodrug, whereby those additive and synergistic combinations are useful in treating mammals, including humans, suffering from atherosclerosis and/or diabetic complications such as, inter alia, diabetic neuropathy, diabetic nephropathy, diabetic cardiomyopathy, diabetic retinopathy, diabetic microangiopathy, diabetic macroangiopathy, cataracts or foot ulcers.

### BACKGROUND OF THE INVENTION

The conversion of 3-hydroxy-3-methylglutaryl-coenzyme A (HMG-CoA) to mevalonate is an early and rate-limiting step in the cholesterol biosynthetic pathway. This step is catalyzed by the enzyme HMG-CoA reductase. Statins inhibit HMG-CoA reductase from catalyzing this conversion. As such, statins are collectively potent lipid lowering agents. Statins include such compounds as atorvastatin, disclosed in U.S. Pat. No. 4,681,893; atorvastatin calcium, disclosed in U.S. Pat. No. 5,273,995; simvastatin, disclosed in U.S. Pat. No. 4,444,784; pravastatin, disclosed in U.S. Pat. No. 4,346,227; cerivastatin, disclosed in U.S. Pat. No. 5,502,199; mevastatin, disclosed in U.S. Pat. No. 3,983,140; velostatin, disclosed in U.S. Pat. No. 4,448,784 and U.S. Pat. No. 4,450,171; fluvastatin, disclosed in U.S. Pat. No. 4,739,073; compactin, disclosed in U.S. Pat. No. 4,804,770; lovastatin, disclosed in U.S. Pat. No. 4,231,938; dalvastatin, disclosed in European Patent Application Publication No. 738510 A2; fluindostatin, disclosed in European Patent Application Publication No. 363934 A1; and dihydrocompactin, disclosed in U.S. Pat. No. 4,450,171. Each of the above is incorporated herein by reference.

Atorvastatin calcium, disclosed in U.S. Pat. No. 5,273,995, which is incorporated herein by reference, is currently sold as Lipitor® and has the formula

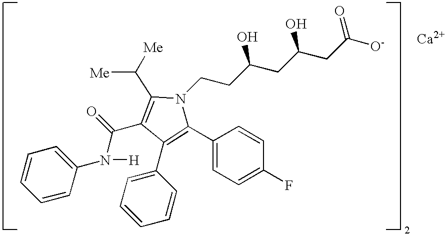

Atorvastatin calcium is a selective, competitive inhibitor of HMG-CoA. As such, atorvastatin calcium is a potent lipid lowering compound. The free carboxylic acid form of atorvastatin exists predominantly as the lactone of the formula

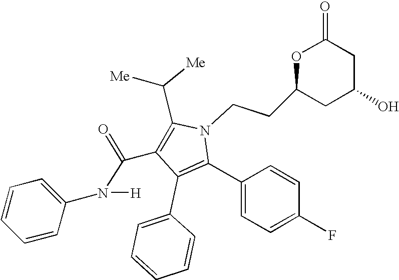

and is disclosed in U.S. Pat. No. 4,681,893, which is incorporated herein by reference.

Atherosclerosis is a condition characterized by irregularly distributed lipid deposits in the intima of arteries, including coronary, carotid and peripheral arteries. Atherosclerotic coronary heart disease (hereinafter termed “CHD”) accounts for 53% of all deaths attributable to a cardiovascular event. CHD accounts for nearly one-half (about $50-60 billion) of the total U.S. cardiovascular healthcare expenditures and about 6% of the overall United States national medical care debt each year. Despite attempts to modify secondary risk factors such as, inter alia, smoking, obesity and lack of exercise, and treatment of dyslipidemia with dietary modification and drug therapy, CHD remains the most common cause of death in the United States.

High levels of blood cholesterol and blood lipids are conditions involved in the onset of atherosclerosis. It is well known that inhibitors of 3-hydroxy-3-methylglutaryl-coenzyme A reductase (HMG-CoA reductase) are effective in lowering the level of blood plasma cholesterol, especially low density lipoprotein cholesterol (LDL-C), in man (Brown and Goldstein, New England Journal of Medicine, 1981, 305, No. 9, 515-517). It has now been established that lowering LDL-C levels affords protection from coronary heart disease (see, e.g., The Scandinavian Simvastatin Survival Study Group: Randomised trial of cholesterol lowering in 4444 patients with coronary heart disease: the Scandinavian Simvastatin Survival Study (4S), Lancet, 1994, 344, 1383-89; and Shepherd, J. et al., Prevention of coronary heart disease with pravastatin in men with hypercholesterolemia, New England Journal of Medicine, 1995, 333, 1301-07).

S. Ao et al., Metabolism, 40, 77-87 (1991) have shown that significant functional improvement in the nerves of diabetic rats (based on nerve conduction velocity) occurs when nerve fructose levels are pharmacologically lowered, and that such improvement correlates more closely with the lowering of nerve fructose than the lowering of nerve sorbitol. Similar results were reported by N. E. Cameron and M. A. Cotter, Diabetic Medicine, 8, Suppl. 1, 35A-36A (1991). In both of these cases, lowering of nerve fructose was achieved using relatively high does of aldose reductase inhibitors, which inhibit the formation of sorbitol, a precursor of fructose, from glucose via the enzyme aldose reductase.

Commonly assigned U.S. Pat. Nos. 5,728,704 and 5,866,578, which are hereby incorporated by reference, each disclose compounds of the Formula A,

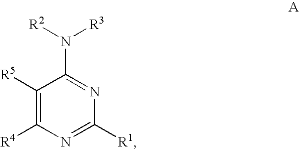

wherein R¹ through R⁵ in the compound of Formula A are defined as disclosed therein. Further, U.S. Pat. No. 5,728,704 discloses that sorbitol dehydrogenase compounds have utility in the treatment of diabetic complications.

Commonly assigned U.S. patent application Ser. No. 09/538,039, which is incorporated herein by reference, discloses compounds of the formula

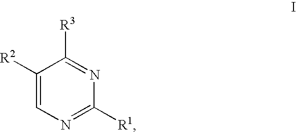

wherein R¹ through R³ in the compound of Formula I are defined as disclosed therein. That application discloses that the compounds of Formula I have utility in the treatment of diabetic complications.

International Patent Application Publication Number WO00/45818 discloses the use of a combination of a statin and an aldose reductase inhibitor for the manufacture of a medicament for the treatment of diabetic neuropathy.

### SUMMARY OF THE INVENTION

This invention is directed to pharmaceutical compositions comprising:

a. an amount of a statin, a prodrug thereof or a pharmaceutically acceptable salt of said statin or said prodrug;

b. an amount of a sorbitol dehydrogenase inhibitor, a prodrug thereof or a pharmaceutically acceptable salt of said sorbitol dehydrogenase inhibitor or said prodrug; and, optionally,

c. a pharmaceutically acceptable carrier or diluent.

This invention is also particularly directed to such pharmaceutical compositions wherein said sorbitol dehydrogenase inhibitor is a compound of Formula I,

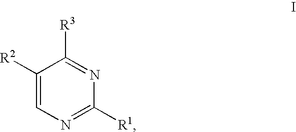

a prodrug thereof or a pharmaceutically acceptable salt of said compound or said prodrug, wherein:

R¹ is formyl, acetyl, propionyl, carbamoyl or —C(OH)R⁴R⁵;

R¹ and R⁵ are each independently hydrogen, methyl, ethyl or hydroxy-(C₁-C₃)alkyl;

R² is hydrogen, (C₁-C₄)alkyl or (C₁-C₄)alkoxy;

R³ is a radical of the formula

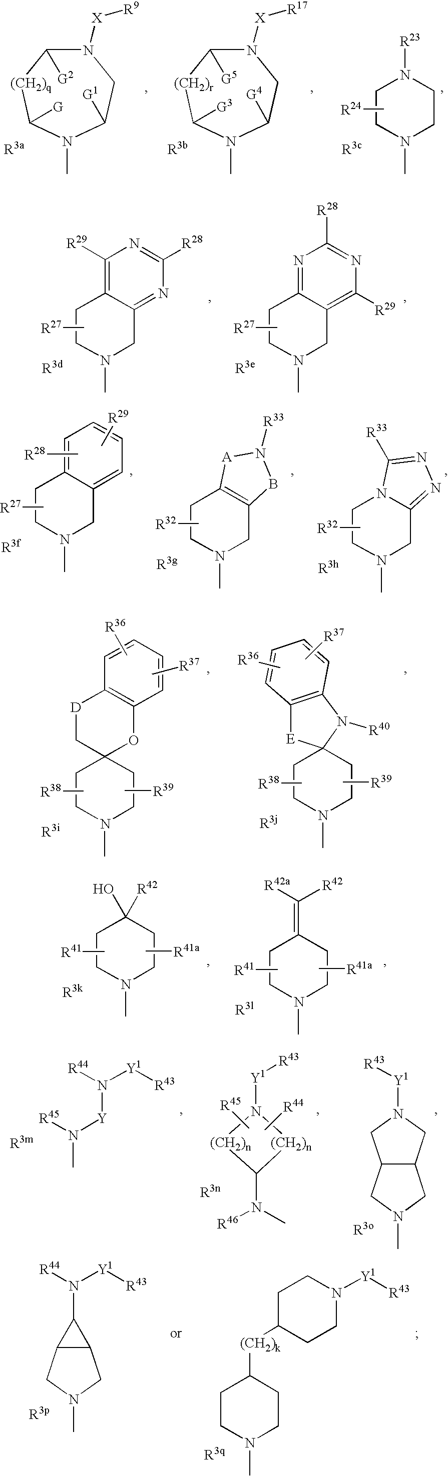

wherein said radical of formula R^{3a} is additionally substituted on the ring by R⁶, R⁷ and R⁸;

said radical of formula R^{3b} is additionally substituted on the ring by R¹⁸, R¹⁹ and R²⁰;

G, G¹ and G² are taken separately and are each hydrogen and R⁶ is hydrogen, (C₁-C₄)alkyl, (C₁-C₄)alkoxycarbonyl, (C₁-C₄)alkoxy-(C₁-C₄)alkyl, hydroxy-(C₁-C₄)alkyl or phenyl optionally independently substituted with up to three hydroxy, halo, hydroxy-(C₁-C₄)alkyl, (C₁-C₄)alkoxy-(C₁-C₄)alkyl, (C₁-C₄)alkyl or (C₁-C₄)alkoxy, wherein said (C₁-C₄)alkyl in the definition of R⁶ and said (C₁-C₄)alkoxy in the definition of R⁶ are optionally and independently substituted with up to five fluoro; R⁷ and R⁸ are each independently hydrogen or (C₁-C₄)alkyl; or

G and G¹ are taken together and are (C₁-C₃)alkylene and R⁶, R⁷, R⁸ and G² are hydrogen; or

G¹ and G² are taken together and are (C₁-C₃)alkylene and R⁶, R⁷, R⁸ and G are hydrogen;

q is 0 or 1;

X is a covalent bond, —(C═NR¹⁰)—, oxycarbonyl, vinylenylcarbonyl, oxy(C₁-C₄)alkylenylcarbonyl, (C₁-C₄)alkylenylcarbonyl, (C₃-C₄)alkenylcarbonyl, thio(C₁-C₄)alkylenylcarbonyl, vinylenylsulfonyl, sulfinyl-(C₁-C₄)alkylenylcarbonyl, sulfonyl-(C₁-C₄)alkylenylcarbonyl or carbonyl(C₀-C₄)alkylenylcarbonyl; wherein said oxy(C₁-C₄)alkylenylcarbonyl, (C₁-C₄)alkylenylcarbonyl, (C₃-C₄)alkenylcarbonyl and thio(C₁-C₄)alkylenylcarbonyl in the definition of X are each optionally and independently substituted with up to two (C₁-C₄)alkyl, benzyl or Ar; said vinylenylsulfonyl and said vinylenylcarbonyl in the definition of X are optionally substituted independently on one or two vinylenyl carbons with (C₁-C₄)alkyl, benzyl or Ar; and said carbonyl(C₀-C₄)alkylenylcarbonyl in the definition of X is optionally substituted indepedently with up to three (C₁-C₄)alkyl, benzyl or Ar;

R¹⁰ is hydrogen or (C₁-C₄)alkyl;

R⁹ is (C₃-C₇)cycloalkyl, Ar¹-(C₀-C₃)alkylenyl or (C₁-C₆)alkyl optionally substituted with up to five fluoro; provided that when q=0 and X is a covalent bond, oxycarbonyl or (C₁-C₄)alkylenylcarbonyl, then R⁹ is not (C₁-C₆)alkyl;

Ar and Ar¹ are independently a fully saturated, partially saturate or fully unsaturated five- to eight-membered ring optionally having up to four heteroatoms selected independently from oxygen, sulfur and nitrogen, or a bicyclic ring consisting of two fused independently partially saturated, fully saturated or fully unsaturated five- to seven-membered rings, taken independently, optionally having up to four heteroatoms selected independently from nitrogen, sulfur and oxygen, or a tricyclic ring consisting of three fused independently partially saturated, fully saturated or fully unsaturated five to seven membered rings, taken independently, optionally having up to four heteroatoms selected independently from nitrogen, sulfur and oxygen, said partially saturated, fully saturated ring or fully unsaturated monocyclic ring, bicyclic ring or tricyclic ring optionally having one or two oxo groups substituted on carbon or one or two oxo groups substituted on sulfur;

Ar and Ar¹ are optionally independently substituted on carbon or nitrogen, on one ring if the moiety is monocyclic, on one or both rings if the moiety is bicyclic, or on one, two or three rings if the moiety is tricyclic, with up to a total of four substituents independently selected from R¹¹, R¹², R¹³ and R¹⁴; wherein R¹¹, R¹², R¹³ and R¹⁴ are each taken separately and are each independently halo, formyl, (C₁-C₆)alkoxycarbonyl, (C₁-C₆)alkylenyloxycarbonyl, (C₁-C₄)alkoxy-(C₁-C₄)alkyl, C(OH)R¹⁵R¹⁶, naphthyl, phenyl, imidazolyl, pyridyl, triazolyl, morpholinyl, (C₀-C₄)alkylsulfamoyl, N-(C₀-C₄)alkylcarbamoyl, N,N-di-(C₁-C₄)alkylcarbamoyl, N-phenylcarbamoyl, N-(C₁-C₄)alkyl-N-phenylcarbamoyl, N,N-diphenyl carbamoyl, (C₁-C₄)alkylcarbonylamido, (C₃-C₇)cycloalkylcarbonylamido, phenylcarbonylamido, piperidinyl, pyrrolidinyl, piperazinyl, cyano, benzimidazolyl, amino, anilino, pyrimidyl, oxazolyl, isoxazolyl, tetrazolyl, thienyl, thiazolyl, benzothiazolyl, pyrrolyl, pyrazolyl, tetrahydroquinolyl, tetrahydroisoquinolyl, benzoxazolyl, pyridazinyl, pyridyloxy, pyridylsulfanyl, furanyl, 8-(C₁-C₄)alkyl-3,8-diaza[3.2.1]bicyclooctyl, 3,5-dioxo-1,2,4-triazinyl, phenoxy, thiophenoxy, (C₁-C₄)alkylsulfanyl, (C₁-C₄)alkylsulfonyl, (C₃-C₇)cycloalkyl, (C₁-C₄)alkyl optionally substituted with up to five fluoro or (C₁-C₄)alkoxy optionally substituted with up to five fluoro; said naphthyl, phenyl, pyridyl, piperidinyl, benzimidazolyl, pyrimidyl, thienyl, benzothiazolyl, pyrrolyl, tetrahydroquinolyl, tetrahydroisoquinolyl, benzoxazolyl, pyridazinyl, pyridyloxy, pyridylsulfanyl, furanyl, thiophenoxy, anilino and phenoxy in the definition of R¹¹, R¹², R¹³ and R¹⁴ are optionally substituted with up to three substituents independently selected from hydroxy, halo, hydroxy-(C₁-C₄)alkyl, (C₁-C₄)alkoxy-(C₁-C₄)alkyl, (C₁-C₄)alkyl optionally substituted with up to five fluoro and (C₁-C₄)alkoxy optionally substituted with up to five fluoro; said imidazolyl, oxazolyl, isoxazolyl, thiazolyl and pyrazolyl in the definition of R¹¹, R¹², R¹³ and R¹⁴ are optionally substituted with up to two substituents independently selected from hydroxy, halo, hydroxy-(C₁-C₄)alkyl, (C₁-C₄)alkoxy-(C₁-C₄)alkyl, (C₁-C₄)alkyl optionally substituted with up to five fluoro and (C₁-C₄)alkoxy optionally substituted with up to five fluoro; said morpholinyl in the definition of R¹¹, R¹², R¹³ and R¹⁴ is optionally substituted with up to two substituents independently selected from (C₁-C₄)alkyl; said pyrrolidinyl in the definition of R¹¹, R¹², R¹³ and R¹⁴ is optionally substituted with up to two substituents independently selected from hydroxy, hydroxy-(C₁-C₃)alkyl, (C₁-C₄)alkoxy-(C₁-C₄)alkyl, (C₁-C₄)alkyl optionally substituted with up to five fluoro and (C₁-C₄)alkoxy optionally substituted with up to five fluoro; said piperazinyl in the definition of R¹¹, R¹², R¹³ and R¹⁴ is optionally substituted with up to three substituents independently selected from (C₁-C₄)alkoxy-(C₁-C₄)alkyl, hydroxy-(C₁-C₃)alkyl, phenyl, pyridyl, (C₀-C₄)alkylsulfamoyl, (C₁-C₄)alkyl optionally substituted with up to five fluoro and (C₁-C₄)alkoxy optionally substituted with up to five fluoro; said triazolyl in the definition of R¹¹, R¹², R¹³ and R¹⁴ is optionally substituted with hydroxy, halo, hydroxy-(C₁-C₄)alkyl, (C₁-C₄)alkoxy-(C₁-C₄)alkyl, (C₁-C₄)alkyl optionally substituted with up to five fluoro and (C₁-C₄)alkoxy optionally substituted with up to five fluoro; said tetrazolyl in the definition of R¹¹, R¹², R¹³ and R¹⁴ is optionally substituted with hydroxy-(C₂-C₃)alkyl or (C₁-C₄)alkyl optionally substituted with up to five fluoro; and said phenyl and pyridyl which are optionally substituted on piperazine in the definition of R¹¹, R¹², R¹³ and R¹⁴ are optionally substituted with up to three hydroxy, halo, hydroxy-(C₁-C₄)alkyl, (C₁-C₄)alkoxy-(C₁-C₄)alkyl, (C₁-C₄)alkyl optionally substituted with up to five fluoro and (C₁-C₄)alkoxy optionally substituted with up to five fluoro; or

R¹¹ and R¹² are taken together on adjacent carbon atoms and are —CH₂OC(CH₃)₂OCH₂— or —O—(CH₂)ₚ—O—, and R¹³ and R¹⁴ are taken separately and are each independently hydrogen or (C₁-C₄)alkyl;

p is 1, 2 or 3;

R¹⁵ and R¹⁶ are taken separately and are each independently hydrogen, (C₁-C₄)alkyl optionally substituted with up to five fluoro; or R¹⁵ and R¹⁶ are taken separately and R¹⁵ is hydrogen and R¹⁶ is (C₃-C₆)cycloalkyl, hydroxy-(C₁-C₃)alkyl, phenyl, pyridyl, pyrimidyl, thienyl, furanyl, thiazolyl, oxazolyl, imidazolyl, benzothiazolyl or benzoxazolyl; or R¹⁵ and R¹⁶ are taken together and are (C₃-C₆)alkylene;

G³, G⁴ and G⁵ are taken separately and are each hydrogen; r is 0; R¹⁸ is hydrogen, (C₁-C₄)alkyl, (C₁-C₄)alkoxycarbonyl, (C₁-C₄)alkoxy-(C₁-C₄)alkyl, hydroxy-(C₁-C₄)alkyl or phenyl optionally independently substituted with up to three hydroxy, halo, hydroxy-(C₁-C₄)alkyl, (C₁-C₄)alkoxy-(C₁-C₄)alkyl, (C₁-C₄)alkyl or (C₁-C₄)alkoxy, wherein said (C₁-C₄)alkyl in the definition of R⁶ and said (C₁-C₄)alkoxy in the definition of R⁶ are optionally and independently substituted with up to five fluoro; and R¹⁹ and R²⁰ are each independently (C₁-C₄)alkyl; or

G³, G⁴ and G⁵ are taken separately and are each hydrogen; r is 1; R¹⁸ is hydrogen, (C₁-C₄)alkyl, (C₁-C₄)alkoxycarbonyl, (C₁-C₄)alkoxy-(C₁-C₄)alkyl, hydroxy-(C₁-C₄)alkyl or phenyl optionally independently substituted with up to three hydroxy, halo, hydroxy-(C₁-C₄)alkyl, (C₁-C₄)alkoxy-(C₁-C₄)alkyl, (C₁-C₄)alkyl or (C₁-C₄)alkoxy, wherein said (C₁-C₄)alkyl in the definition of R⁶ and said (C₁-C₄)alkoxy in the definition of R⁶ are optionally and independently substituted with up to five fluoro; and R¹⁹ and R²⁰ are each independently hydrogen or (C₁-C₄)alkyl; or

G³ and G⁴ are taken together and are (C₁-C₃)alkylene; r is 0 or 1; and R¹⁸, R¹⁹, R²⁰ and G⁵ are hydrogen; or

G⁴ and G⁵ are taken together and are (C₁-C₃)alkylene; r is 0 or 1; and R¹⁸, R¹⁹, R²⁰ and G³ are hydrogen;

R¹⁷ is SO₂NR²¹R²², CONR²¹R²², (C₁-C₆)alkoxycarbonyl, (C₁-C₆)alkylcarbonyl, Ar²-carbonyl, (C₁-C₆)alkylsulfonyl, (C₁-C₆)alkylsulfinyl, Ar²-sulfonyl, Ar²-sufinyl and (C₁-C₆)alkyl;

R²¹ and R²² are taken separately and are each independently selected from hydrogen, (C₁-C₆)alkyl, (C₃-C₇)cycloalkyl and Ar²—(C₀-C₄)alkylenyl; or

R²¹ and R²² are taken together with the nitrogen atom to which they are attached to form azetidinyl, pyrrolidinyl, piperidinyl, piperazinyl, morpholinyl, azepinyl, azabicyclo[3.2.2]nonanyl, azabicyclo[2.2.1]heptyl, 6,7-dihydro-5H-dibenzo[c,e]azepinyl, 1,2,3,4-tetrahydro-isoquinolyl or 5,6,7,8-tetrahydropyrido[4,3-d]pyrimidyl; said azetidinyl in the definition of R²¹ and R²² is optionally substituted independently with one substituent selected from hydroxy, amino, hydroxy-(C₁-C₄)alkyl, (C₁-C₄)alkoxy-(C₁-C₄)alkyl, (C₁-C₄)alkyl optionally substituted with up to five fluoro and (C₁-C₄)alkoxy optionally substituted with up to five fluoro; said pyrrolidinyl, piperidinyl, azepinyl in the definition of R²¹ and R²² are optionally substituted independently with up to two substituents independently selected from hydroxy, amino, hydroxy-(C₁-C₄)alkyl, (C₁-C₄)alkoxy-(C₁-C₄)alkyl, (C₁-C₄)alkyl optionally substituted with up to five fluoro and (C₁-C₄)alkoxy optionally substituted with up to five fluoro; said morpholinyl in the definition of R²¹ and R²² is optionally substituted with up to two substituents independently selected from hydroxy-(C₁-C₄)alkyl, (C₁-C₄)alkoxy-(C₁-C₄)alkyl, (C₁-C₄)alkyl optionally substituted with up to five fluoro and (C₁-C₄)alkoxy optionally substituted with up to five fluoro; said piperazinyl in the definition of R²¹ and R²² is optionally substituted independently with up to three substituents independently selected from phenyl, pyridyl, (C₁-C₄)alkoxycarbonyl and (C₁-C₄)alkyl optionally substituted with up to five fluoro; said 1,2,3,4-tetrahydroisoquinolyl and said 5,6,7,8-tetrahydropyrido[4,3-d]pyrimidyl in the definition of R²¹ and R²² are optionally substituted independently with up to three substituents independently selected from hydroxy, amino, halo, hydroxy-(C₁-C₄)alkyl, (C₁-C₄)alkoxy-(C₁-C₄)alkyl, (C₁-C₄)alkyl optionally substituted with up to five fluoro and (C₁-C₄)alkoxy optionally substituted with up to five fluoro; and said 6,7-dihydro-5H-dibenzo[c,e]azepinyl in the definition of R²¹ and R²² is optionally substituted with up to four substituents independently selected from hydroxy, amino, halo, hydroxy-(C₁-C₄)alkyl, (C₁-C₄)alkoxy-(C₁-C₄)alkyl, (C₁-C₄)alkyl optionally substituted with up to five fluoro and (C₁-C₄)alkoxy optionally substituted with up to five fluoro; said pyrimidyl, pyridyl and phenyl which are optionally substituted on said piperazine in the definition of R²¹ and R²² is optionally substituted with up to three substituents selected from hydroxy, amino, hydroxy-(C₁-C₄)alkyl, (C₁-C₄)alkoxy-(C₁-C₄)alkyl, (C₁-C₄)alkyl optionally substituted with up to five fluoro and (C₁-C₄)alkoxy optionally substituted with up to five fluoro;

Ar² is independently defined as set forth for Ar and Ar¹ above;

said Ar² is optionally independently substituted as set forth for Ar and Ar¹ above;

R²³ is CONR²⁵R²⁶ or SO₂R²⁵R²⁶, wherein R²⁵ is hydrogen (C₁-C₄)alkyl or Ar³—(C₀-C₄)alkylenyl and R²⁶ is Ar³—(C₀-C₄)alkylenyl; provided that when Ar³ is phenyl, naphthyl or biphenyl, then R²³ cannot be CONR²⁵R²⁶ where R²⁵ is hydrogen or Ar³ and R²⁶ is Ar³;

R²⁴ is hydrogen, (C₁-C₄)alkyl, (C₁-C₄)alkoxycarbonyl, (C₁-C₄)alkoxy-(C₁-C₄)alkyl, hydroxy-(C₁-C₄)alkyl or phenyl optionally independently substituted with up to three hydroxy, halo, hydroxy-(C₁-C₄)alkyl, (C₁-C₄)alkoxy-(C₁-C₄)alkyl, (C₁-C₄)alkyl or (C₁-C₄)alkoxy, wherein said (C₁-C₄)alkyl in the definition of R⁶ and said (C₁-C₄)alkoxy in the definition of R⁶ are optionally and independently substituted with up to five fluoro;

Ar³ is independently defined as set forth for Ar and Ar¹ above;

said Ar³ is optionally independently substituted as set forth for Ar and Ar¹ above;

R²⁷ is hydrogen or (C₁-C₄)alkyl;

R²⁸ and R²⁹ are each independently hydrogen, hydroxy, halo, hydroxy-(C₁-C₄)alkyl, (C₁-C₄)alkoxy-(C₁-C₄)alkyl, (C₁-C₄)alkyl optionally substituted with up to five fluoro, (C₁-C₄)alkoxy optionally substituted with up to five fluoro, phenyl, pyridyl, pyrimidyl, thienyl, furanyl, thiazolyl, oxazolyl, phenoxy, thiophenoxy, SO₂NR³⁰R³¹, CONR³⁰R³¹ or NR³⁰R³¹; said thienyl, pyrimidyl, furanyl, thiazolyl and oxazolyl in the definition of R²⁸ and R²⁹ are optionally substituted by up to two hydroxy, halo, hydroxy-(C₁-C₄)alkyl, (C₁-C₄)alkoxy-(C₁-C₄)alkyl, (C₁-C₄)alkyl optionally substituted with up to five fluoro or (C₁-C₄)alkoxy optionally substituted with up to five fluoro; said phenyl, pyridyl, phenoxy and thiophenoxy in the definition of R²⁸ and R²⁹ are optionally substituted by up to three hydroxy, halo, hydroxy-(C₁-C₄)alkyl, (C₁-C₄)alkoxy-(C₁-C₄)alkyl, (C₁-C₄)alkyl optionally substituted with up to five fluoro or (C₁-C₄)alkoxy optionally substituted with up to five fluoro;

R³⁰ and R³¹ are each independently hydrogen, (C₁-C₄)alkyl, (C₃-C₇)cycloalkyl or phenyl, said phenyl is optionally substituted with up to three hydroxy, halo, hydroxy-(C₁-C₄)alkyl, (C₁-C₄)alkoxy-(C₁-C₄)alkyl, (C₁-C₄)alkyl optionally substituted with up to five fluoro or (C₁-C₄)alkoxy optionally substituted with up to five fluoro; or

R³⁰ and R³¹ are taken together with the nitrogen to which they are attached to form indolinyl, pyrrolidinyl, piperidinyl, piperazinyl or morpholinyl; said pyrrolidinyl and piperidinyl in the definition of R³⁰ and R³¹ are optionally substituted with up to two hydroxy, amino, hydroxy-(C₁-C₄)alkyl, (C₁-C₄)alkoxy-(C₁-C₄)alkyl, (C₁-C₄)alkyl optionally substituted with up to five fluoro or (C₁-C₄)alkoxy optionally substituted with up to five fluoro; said indolinyl and piperazinyl in the definition of R³⁰ and R³¹ are optionally substituted with up to three hydroxy, amino, hydroxy-(C₁-C₄)alkyl, (C₁-C₄)alkoxy-(C₁-C₄)alkyl, (C₁-C₄)alkoxycarbonyl, (C₁-C₄)alkyl optionally substituted with up to five fluoro or (C₁-C₄)alkoxy optionally substituted with up to five fluoro; said morpholinyl in the definition of R³⁰ and R³¹ is optionally substituted with up to two substituents independently selected from hydroxy-(C₁-C₄)alkyl, (C₁-C₄)alkoxy-(C₁-C₄)alkyl, (C₁-C₄)alkyl optionally substituted with up to five fluoro and (C₁-C₄)alkoxy optionally substituted with up to five fluoro;

A is N optionally substituted with hydrogen or (C₁-C₄)alkyl and B is carbonyl; or

A is carbonyl and B is N optionally substituted with hydrogen or (C₁-C₄)alkyl;

R³² is hydrogen or (C₁-C₄)alkyl;

R³³ is phenyl, pyridyl, pyrimidyl, thiazolyl, oxazolyl, benzyl, quinolyl, isoquinolyl, phthalizinyl, quinoxanlyl, benzothiazoyl, benzoxazolyl, benzofuranyl or benzothienyl;

said phenyl, pyridyl, pyrimidyl, thiazolyl, oxazolyl, benzyl, quinolyl, isoquinolyl, phthalizinyl, quinoxanlyl, benzothiazoyl, benzoxazolyl, benzofuranyl and benzothienyl in the definition of R³³ are optionally substituted with up to three phenyl, phenoxy, NR³⁴R³⁵, halo, hydroxy, hydroxy-(C₁-C₄)alkyl, (C₁-C₄)alkoxy-(C₁-C₄)alkyl, (C₁-C₄)alkyl optionally substituted with up to five fluoro or (C₁-C₄)alkoxy optionally substituted with up to five fluoro;

R³⁴ and R³⁵ are each independently hydrogen, (C₁-C₄ alkyl), phenyl or phenylsulfonyl;

said phenyl and phenylsulfonyl in the definition of R³⁴ and R³⁵ are optionally substituted with up to three halo, hydroxy, (C₁-C₄)alkyl optionally substituted with up to five fluoro or (C₁-C₄)alkoxy optionally substituted with up to five fluoro;

D is CO, CHOH or CH₂;

E is O, NH or S;

R³⁶ and R³⁷ are taken separately and are each independently hydrogen, halo, cyano, hydroxy, amino, (C₁-C₆)alkylamino, di-(C₁-C₆)alkylamino, pyrrolidino, piperidino, morpholino, (C₁-C₄)alkoxy-(C₁-C₄)alkyl, hydroxy-(C₁-C₄)alkyl, Ar⁴, (C₁-C₄)alkyl optionally substituted with up to five fluoro or (C₁-C₄)alkoxy optionally substituted with up to five fluoro;

R³⁸, R³⁹ and R⁴⁰ are each independently hydrogen or (C₁-C₄)-alkyl;

Ar⁴ is phenyl, furanyl, thienyl, pyridyl, pyrimidyl, pyrazinyl or pyridazinyl; said Ar⁴ being optionally substituted with up to three hydroxy, (C₁-C₄)alkoxy-(C₁-C₄)alkyl, halo, hydroxy-(C₁-C₄)alkyl, (C₁-C₄)alkyl optionally substituted with up to five fluoro or (C₁-C₄)alkoxy optionally substituted with up to five fluoro; or

R³⁶ and R³⁷ are taken together on adjacent carbon atoms and are —O—(CH₂)ₜ—O—;

t is 1, 2 or 3;

Y is (C₂-C₆)alkylene;

R⁴⁴, R⁴⁵ and R⁴⁶ are each independently hydrogen or (C₁-C₄)alkyl;

m and n are each independently 1, 2 or 3, provided that the sum of m and n is 2, 3 or 4;

k is 0, 1, 2, 3 or 4;

Y¹ is a covalent bond, carbonyl, sulfonyl or oxycarbonyl;

R⁴³ is (C₃-C₇)cycloalkyl, Ar⁵—(C₀-C₄)alkylenyl, NR⁴⁷R⁴⁸ or (C₁-C₆)alkyl optionally substituted with one to five fluoro; provided that when Y¹ is a covalent bond or oxycarbonyl, then R⁴³ is not NR⁴⁷R⁴⁸;

R⁴⁷ and R⁴⁸ are taken separately and are each independently selected from hydrogen, Ar⁵, (C₁-C₆)alkyl and Ar⁵—(C₀-C₄)alkylenyl; or

R⁴⁷ and R⁴⁸ are taken together with the nitrogen atom to which they are attached to form azetidinyl, pyrrolidinyl, piperidinyl, piperazinyl, morpholinyl, azepinyl, azabicyclo[3.2.2]nonanyl, azabicyclo[2.2.1]heptyl, 1,2,3,4-tetrahydroisoquinolyl, 6,7-dihydro-5H-dibenzo[c,e]azepinyl or 5,6,7,8-tetrahydropyrido[4,3-d]pyrimidyl; said azetidinyl in the definition of R⁴⁷ and R⁴⁸ are optionally substituted with one hydroxy, amino, hydroxy-(C₁-C₄)alkyl, (C₁-C₄)alkoxy-(C₁-C₄)alkyl, (C₁-C₄)alkyl optionally substituted with up to five fluoro or (C₁-C₄)alkoxy optionally substituted with up to five fluoro; said pyrrolidinyl, piperidinyl and azepinyl in the definition of R⁴⁷ and R⁴⁸ are optionally substituted with up to two hydroxy, amino, hydroxy-(C₁-C₄)alkyl, (C₁-C₄)alkoxy-(C₁-C₄)alkyl, (C₁-C₄)alkyl optionally substituted with up to five fluoro or (C₁-C₄)alkoxy optionally substituted with up to five fluoro; said morpholinyl in the definition of R⁴⁷ and R⁴⁸ is optionally substituted with up to two substituents independently selected from hydroxy-(C₁-C₄)alkyl, (C₁-C₄)alkoxy-(C₁-C₄)alkyl, (C₁-C₄)alkyl optionally substituted with up to five fluoro and (C₁-C₄)alkoxy optionally substituted with up to five fluoro; said piperazinyl, 1,2,3,4-tetrahydroisoquinolyl and 5,6,7,8-tetrahydro[4,3-d]pyrimidyl in the definition of R⁴⁷ and R⁴⁸ are optionally substituted with up to three hydroxy, amino, halo, hydroxy-(C₁-C₄)alkyl, (C₁-C₄)alkoxy-(C₁-C₄)alkyl, (C₁-C₄)alkyl optionally substituted with up to five fluoro or (C₁-C₄)alkoxy optionally substituted with up to five fluoro; and said 6,7-dihydro-5H-dibenzo[c,e]azepinyl in the definition of R⁴⁷ and R⁴⁸ are optionally substituted with up to four hydroxy, amino, halo, hydroxy-(C₁-C₄)alkyl, (C₁-C₄)alkoxy-(C₁-C₄)alkyl, (C₁-C₄)alkyl optionally substituted with up to five fluoro or (C₁-C₄)alkoxy optionally substituted with up to five fluoro;

Ar⁵ is independently defined as set forth for Ar and Ar¹ above;

Ar⁵ is optionally independently substituted as set forth for Ar and Ar¹ above;

R⁴² and R^{42a} are independently hydrogen, (C₃-C₇)cycloalkyl, Ar⁶—(C₀-C₃)alkylenyl, —(C₂-C₄)alkenyl, Ar⁶-carbonyl or (C₁-C₆)alkyl optionally substituted with up to five fluoro;

Ar⁶ is independently defined as set forth for Ar and Ar¹ above;

Ar⁶ is optionally independently substituted as set forth for Ar and Ar¹ above; and

R⁴¹ and R^{41a} are each independently hydrogen or (C₁-C₄)alkyl.

This invention is more particularly directed to such pharmaceutical compositions of the immediately preceding paragraph, wherein in said compound of Formula I, said prodrug thereof or said pharmaceutically acceptable salt of said compound or said prodrug:

R¹ is C(OH)R⁴R⁵, where R⁴ and R⁵ are each independently hydrogen or methyl;

R² is hydrogen;

R³ is

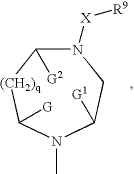

wherein said R³ is substituted by R⁶, R⁷ or R⁸;

G, G¹ and G² are taken separately and are each hydrogen and R⁶ is hydrogen or (C₁-C₄)alkyl; R⁷ and R⁸ are each independently hydrogen or (C₁-C₄)alkyl; or

G and G¹ are taken together and are (C₁-C₃)alkylene and R⁶, R⁷, R⁸ and G² are hydrogen; or

G¹ and G² are taken together and are (C₁-C₃)alkylene and R⁶, R⁷, R⁸ and G are hydrogen;

q is 0 or 1;

X is a covalent bond, oxycarbonyl, vinylenylcarbonyl, oxy(C₁-C₄)alkylenylcarbonyl, thio(C₁-C₄)alkylenylcarbonyl or vinylenylsulfonyl; said vinylenylcarbonyl and said vinylenylsulfonyl in the definition of X are optionally substituted on one or two vinylenyl carbons with (C₁-C₄)alkyl, benzyl or Ar; said oxy(C₁-C₄)alkylenylcarbonyl and said thio(C₁-C₄)alkylenylcarbonyl in the definition of X are optionally substituted with up to two (C₁-C₄)alkyl, benzyl or Ar;

R⁹ is (C₃-C₇)cycloalkyl, Ar¹—(C₀-C₄)alkylenyl or (C₁-C₆)alkyl optionally substituted with up to five fluoro;

Ar¹ is phenyl, naphthyl, pyridyl, pyrimidyl, pyrazinyl, pyridazinyl, triazinyl, quinolyl, isoquinolyl, quinazolyl, quinoxalyl, phthalazinyl, cinnolinyl, naphthyridinyl, pteridinyl, pyrazinopyrazinyl, pyrazinopyridazinyl, pyrimidopyridazinyl, pyrimidopyrimidyl, pyridopyrimidyl, pyridopyrazinyl, pyridopyridazinyl, pyrrolyl, furanyl, thienyl, imidazolyl, oxazolyl, thiazolyl, pyrazolyl, isoxazolyl, isothiazolyl, triazolyl, oxadiazolyl, thiadiazolyl, tetrazolyl, indolyl, benzofuranyl, benzothienyl, benzimidazolyl, benzoxazolyl, benzothiazolyl, indazolyl, benzisoxazolyl, benzisothiazolyl, pyrrolopyridyl, furopyridyl, thienopyridyl, imidazolopyridyl, oxazolopyridyl, thiazolopyridyl, pyrazolopyridyl, isoxazolopyridyl, isothiazolopyridyl, pyrrolopyrimidyl, furopyrimidyl, thienopyrimidyl, imidazolopyrimidyl, oxazolopyrimidyl, thiazolopyrimidyl, pyrazolopyrimidyl, isoxazolopyrimidyl, isothiazolopyrimidyl, pyrrolopyrazinyl, furopyrazinyl, thienopyrazinyl, imidazolopyrazinyl, oxazolopyrazinyl, thiazolopyrazinyl, pyrazolopyrazinyl, isoxazolopyrazinyl, isothiazolopyrazinyl, pyrrolopyridazinyl, furopyridazinyl, thienopyridazinyl, imidazolopyridazinyl, oxazolopyridazinyl, thiazolopyridazinyl, pyrazolopyridazinyl, isoxazolopyridazinyl or isothiazolopyridazinyl; and

said Ar¹ is optionally substituted as set forth above in the definition of the variables of the compounds of Formula I.

This invention is still more particularly directed to such pharmaceutical compositions as set forth in the immediately preceding paragraph, wherein in said compound of Formula I, said prodrug thereof or said pharmaceutically acceptable salt of said compound or said prodrug:

X is a covalent bond, oxycarbonyl or vinylenylcarbonyl optionally substituted on one or two vinylenyl carbons with (C₁-C₄)alkyl, benzyl or Ar;

R⁹ is Ar¹—(C₀-C₄)alkylenyl;

Ar¹ is phenyl, naphthyl, pyridyl, pyrimidyl, pyrazinyl, triazinyl, quinolyl, isoquinolyl, quinazolyl, quinoxalyl, furanyl, thienyl, indolyl, benzofuranyl, benzothienyl, benzoxazolyl, benzothiazolyl, furopyridyl, oxazolopyridyl, thiazolopyridyl, thienopyridyl, furopyrimidyl, thienopyrimidyl, oxazolopyrimidyl or thiazolopyrimidyl; and

said Ar¹ is optionally substituted as set forth above in the definition of the variables of the compounds of Formula I.

This invention is still more particularly directed to such pharmaceutical compositions as set forth in the immediately preceding paragraph wherein in said compound of Formula I, said prodrug thereof or said pharmaceutically acceptable salt of said compound or said prodrug:

R² is hydrogen;

R⁴ is hydrogen or methyl;

R⁵ is methyl;

G, G¹ and G² are hydrogen;

R⁶ and R⁷ are each independently hydrogen or methyl;

R⁸ is hydrogen.

Preferred sorbitol dehydrogenase inhibitors for use in the pharmaceutical compositions, methods and kits of this invention include:

R-(4-{1′-[2-(1R-hydroxy-ethyl)-pyrimidin-4-yl]-[4,4′]bipiperidinyl-1-yl}-pyrimidin-2-yl)-ethanol;

furo[3,2-c]pyridin-2-yl-{4-[2-(1R-hydroxy-ethyl)-pyrimidin-4-yl]-3R,5S-dimethyl-piperazin-1-yl}-methanone;

(4-chloro-furo[3,2-c]pyridin-2-yl)-{4-[2-(1R-hydroxy-ethyl)-pyrimidin-4-yl]-3R,5S-dimethyl-piperazin-1-yl}-methanone;

{4-[2-(1R-hydroxy-ethyl)-pyrimidin-4-yl]-3R,5S-dimethyl-piperazin-1-yl}-(4-pyrrolidin-1-yl-furo[3,2-c]pyridin-2-yl)-methanone;

{4-[2-(1R-hydroxy-ethyl)-pyrimidin-4-yl]-3R,5S-dimethyl-piperazin-1-yl}-(4-morpholin-4-yl-furo[3,2-c]pyridin-2-yl)-methanone;

{4-[2-(1R-hydroxy-ethyl)-pyrimidin-4-yl]-3R,5S-dimethyl-piperazin-1-yl}-imidazo[1,2-a]pyridin-2-yl-methanone;

furo[3,2-c]pyridin-2-yl-{4-[2-(1R-hydroxy-ethyl)-pyrimidin-4-yl]-2R,6S-dimethyl-piperazin-1-yl}-methanone;

4-[2-(1R-hydroxy-ethyl)-pyrimidin-4-yl]-2R,6S-dimethyl-piperazine-1-carboxylic acid pyridin-3-yl ester;

4-[2-(1R-hydroxy-ethyl)-pyrimidin-4-yl]-2R,6S-dimethyl-piperazine-1-carboxylic acid 2-methyl-pyridin-3-yl ester;

4-[2-(1R-hydroxy-ethyl)-pyrimidin-4-yl]-2R,6S-dimethyl-piperazine-1-carboxylic acid 5-chloro-pyridin-3-yl ester;

4-[2-(1R-hydroxy-ethyl)-pyrimidin-4-yl]-2R,6S-dimethyl-piperazine-1-carboxylic acid 6-methyl-pyridin-3-yl ester;

(E)-1-{4-[2-(1R-hydroxy-ethyl)-pyrimidin-4-yl]-2R,6S-dimethyl-piperazin-1-yl}-3-thiophen-2-yl-propenone;

1R-{4-[4-(4,6-dimethyl-pyrimidin-2-yl)-3R,5S-dimethyl-piperazin-1-yl]-pyrimidin-2-yl}-ethanol;

1R-{4-[4-(4-methoxymethyl-6-methyl-pyrimidin-2-yl)-3R,5S-dimethyl-piperazin-1-yl]-pyrimidin-2-yl}-ethanol;

1R-{4-[4-(4-hydroxymethyl-6-methyl-pyrimidin-2-yl)-3R,5S-dimethyl-piperazin-1-yl]-pyrimidin-2-yl}-ethanol;

1R-(4-{3R,5S-dimethyl-4-[2-(4-methyl-piperazin-1-yl)-pyrimidin-4-yl]-piperazin-1-yl}-pyrimidin-2-yl)-ethanol;

1R-(4-{4-[2-(4-ethyl-piperazin-1-yl)-pyrimidin-4-yl]-3R,5S-dimethyl-piperazin-1-yl}-pyrimidin-2-yl)-ethanol;

1R-(4-{3R,5S-dimethyl-4-[2-(4-methyl-imidazol-1-yl)-pyrimidin-4-yl]-piperazin-1-yl}-pyrimidin-2-yl)-ethanol;

1R-(4-{3R,5S-dimethyl-4-[2-(2-methyl-imidazol-1-yl)-pyrimidin-4-yl]-piperazin-1-yl}-pyrimidin-2-yl)-ethanol;

1R-(4-{4-[2-(2,4-dimethyl-imidazol-1-yl)-pyrimidin-4-yl]-3R,5S-dimethyl-piperazin-1-yl}-pyrimidin-2-yl)-ethanol;

1R-(4-{4-[2-(4-isopropyl-piperazin-1-yl)-pyrimidin-4-yl]-3R,5S-dimethyl-piperazin-1-}-pyrimidin-2-yl)-ethanol;

1R-(4-{3R,5S-dimethyl-4-[4-methyl-6-(4-methyl-piperazin-1-yl)-[1,3,5]triazin-2-yl]-piperazin-1-yl}-pyrimidin-2-yl)-ethanol;

1R-{4-[4-(4-methoxy-6-methyl-[1,3,5]triazin-2-yl)-3R,5S-dimethyl-piperazin-1-yl]-pyrimidin-2-yl}-ethanol;

1R-{4-[4-(4,6-dimethoxy-[1,3,5]triazin-2-yl)-3R,5S-dimethyl-piperazin-1-yl]-pyrimidin-2-yl}-ethanol;

1R-{4-[4-(4-ethoxy-6-methyl-[1,3,5]triazin-2-yl)-3R,5S-dimethyl-piperazin-1-yl]-pyrimidin-2-yl}-ethanol;

1R-{4-[4-(4-isopropoxy-6-methyl-[1,3,5]triazin-2-yl)-3R,5S-dimethyl-piperazin-1-yl]-pyrimidin-2-yl}-ethanol;

1R-{4-[3R,5S-dimethyl-4-(4-phenyl-[1,3,5]triazin-2-yl)-piperazin-1-yl]-pyrimidin-2-yl}-ethanol;

1R-{4-[4-(4-hydroxymethyl-6-methoxy-[1,3,5]triazin-2-yl)-3R,5S-dimethyl-piperazin-1-yl]-pyrimidin-2-yl}-ethanol;

1R-{4-[4-(4-isopropoxy-6-methoxy-[1,3,5]triazin-2-yl)-3R,5S-dimethyl-piperazin-1-yl]-pyrimidin-2-yl}-ethanol;

1R-{4-[4-(4-isopropyl-[1,3,5]triazin-2-yl)-3R,5S-dimethyl-piperazin-1-yl]-pyrimidin-2-yl}-ethanol;

1R-{4-[4-(4-ethyl-6-methoxy-[1,3,5]triazin-2-yl)-3R,5S-dimethyl-piperazin-1-yl]-pyrimidin-2-yl}-ethanol;

1R-{4-[4-(4,6-dimethyl-pyrimidin-2-yl)-2R,6S-dimethyl-piperazin-1-yl]-pyrimidin-2-yl}-ethanol;

1R-{4-[4-(4-hydroxymethyl-6-methyl-pyrimidin-2-yl)-2R,6S-dimethyl-piperazin-1-yl]-pyrimidin-2-yl}-ethanol;

1R-{4-[2R,6S-dimethyl-4-(4-[1,2,4]triazol-1-yl-pyrimidin-2-yl)-piperazin-1-yl]-pyrimidin-2-yl}-ethanol;

1R-{4-[4-(2,6-dimethyl-pyrimidin-4-yl)-2R,6S-dimethyl-piperazin-1-yl]-pyrimidin-2-yl}-ethanol;

1R-(4-{4-[2-(1R-hydroxy-ethyl)-pyrimidin-4-yl]-2R,6S-dimethyl-piperazin-1-yl}-pyrimidin-2-yl)-ethanol;

1R-{4-[4-(2-hydroxymethyl-6-methyl-pyrimidin-4-yl)-2R,6S-dimethyl-piperazin-1-yl]-pyrimidin-2-yl}-ethanol;

1R-(4-{4-[2-(1S-hydroxy-ethyl)-pyrimidin-4-yl]-2R,6S-dimethyl-piperazin-1-yl}-pyrimidin-2-yl)-ethanol;

1S-(4-{4-[2-(1R-hydroxy-ethyl)-pyrimidin-4-yl]-2R,6S-dimethyl-piperazin-1-yl}-pyrimidin-2-yl)-ethanol;

1-{4-[4-(2-acetyl-pyrimidin-4-yl)-2R,6S-dimethyl-piperazin-1-yl]-pyrimidin-2-yl}-ethanone;

1RS-(4-{4-[2-(1RS-hydroxy-ethyl)-pyrimidin-4-yl]-2R,6S-dimethyl-piperazin-1-yl}-pyrimidin-2-yl)-ethanol;

(4-{4-[2-(1R-hydroxy-ethyl)-pyrimidin-4-yl]-3R,5S-dimethyl-piperazin-1-yl}-pyrimidin-2-yl)-ethanone;

1R-{4-[2R,6S-dimethyl-4-(2-morpholin-4-yl-pyrimidin-4-yl)-piperazin-1-yl]-pyrimidin-2-yl}-ethanol;

1R-(4-{2R,6S-dimethyl-4-[2-(4-methyl-piperazin-1-yl)-pyrimidin-4-yl]-piperazin-1-yl}-pyrimidin-2-yl)-ethanol;

1R-{4-[2R,6S-dimethyl-4-(2-[1,2,4]triazol-1-yl-pyrimidin-4-yl)-piperazin-1-yl]-pyrimidin-2-yl}-ethanol;

1R-(4-{4-[2-(1R-hydroxy-ethyl)-pyrimidin-4-yl]-2R,6R-dimethyl-piperazin-1-yl}-pyrimidin-2-yl)-ethanol;

1R-(4-{4-[2-(4-ethyl-piperazin-1-yl)-pyrimidin-4-yl]-2R,6S-dimethyl-piperazin-1-yl}-pyrimidin-2-yl)-ethanol;

1R-(4-{2R,6S-dimethyl-4-[2-(4-methyl-imidazol-1-yl)-pyrimidin-4-yl]-piperazin-1-yl}-pyrimidin-2-yl)-ethanol;

1R-(4-{4-[2-(2,4-dimethyl-imidazol-1-yl)-pyrimidin-4-yl]-2R,6S-dimethyl-piperazin-1-yl}-pyrimidin-2-yl)-ethanol;

1R-{4-[2R,6S-dimethyl-4-(4-morpholin-4-yl-[1,3,5]triazin-2-yl)-piperazin-1-yl]-pyrimidin-2-yl}-ethanol;

1R-{4-[4-(4-methoxy-6-methyl-[1,3,5]triazin-2-yl)-2R,6S-dimethyl-piperazin-1-yl]-pyrimidin-2-yl}-ethanol;

1R-{4-[4-(4,6-dimethoxy-[1,3,5]triazin-2-yl)-2R,6S-dimethyl-piperazin-1-yl]-pyrimidin-2-yl}-ethanol;

1R-{4-[2R,6S-dimethyl-4-(4-phenyl-[1,3,5]triazin-2-yl)-piperazin-1-yl]-pyrimidin-2-yl}-ethanol;

1R-{4-[4-(4-hydroxymethyl-6-methyl-pyrimidin-2-yl)-3S-methyl-piperazin-1-yl]-pyrimidin-2-yl}-ethanol;

1R-{4-[4-(2-hydroxymethyl-pyrimidin-4-yl)-3S-methyl-piperazin-1-yl]-pyrimidin-2-yl}-ethanol;

1R-{4-[4-(2-hydroxymethyl-6-methyl-pyrimidin-4-yl)-3S-methyl-piperazin-1-yl]-pyrimidin-2-yl}-ethanol;

1R-[4-(3S-methyl-4-oxazolo[5,4b]pyridin-2-yl-piperazin-1-yl)-pyrimidin-2-yl]-ethanol;

1R-[4-(3S-methyl-4-oxazolo[4,5-b]pyridin-2-yl-piperazin-1-yl)-pyrimidin-2-yl]-ethanol;

1R-[4-(3S-methyl-4-quinoxalin-2-yl -piperazin-1-yl)-pyrimidin-2-yl]-ethanol;

1R-{4-[4-(4,6-dimethyl-[1,3,5]triazin-2-yl)-3R,5S-dimethyl-piperazin-1-yl]-pyrimidin-2-yl}-ethanol;

1R-{4-[3R,5S-dimethyl-4-(4-methyl-6-phenyl-[1,3,5]triazin-2-yl)-piperazin-1-yl]pyrimidin-2-yl}-ethanol;

1R-{4-[4-(4-cyclopropyl-[1,3,5]triazin-2-yl)-3R,5S-dimethyl-piperazin-1-yl]-pyrimidin-2-yl}-ethanol;

1R-{4-[4-(4-cyclopropyl-[1,3,5]triazin-2-yl)-2R, 6S-dimethyl-piperazin-1-yl]-pyrimidin-2-yl}-ethanol;

1R-{4-[4-(4,6-dimethyl-[1,3,5]triazin-2-yl)-2R,6S-dimethyl-piperazin-1-yl]-pyrimidin-2-yl}-ethanol;

1R-{4-[4-(4-hydroxymethyl-6-phenyl-[1,3,5]triazin-2-yl)-2R ,6S-dimethyl-piperazin-1-yl]-pyrimidin-2-yl}-ethanol;

1R-{4-[4-(4-methoxy-6-methoxymethyl-[1,3,5]triazin-2-yl)-2R,6S-dimethyl-piperazin-1-yl]-pyrimidin-2-yl}-ethanol;

1R-{4-[2R ,6S-dimethyl-4-(4-methyl-[1,3,5]triazin-2-yl)-piperazin-1-yl]-pyrimidin-2-yl-ethanol;

1-{4-[4-(2-acetyl-pyrimidin-4-yl)-2R*,6S*-dimethyl-piperazin-1-yl]-pyrimidin-2-yl}-ethanone;

1-(-4-{4-[2-(1R-hydroxy-ethyl)-pyrimidin-4-yl]-2R,6S-dimethyl-piperazin-1-yl}-pyrimidin-2-yl)-ethanone;

1R-{4-[4-(4-methoxymethyl-6-phenyl-[1,3,5]-triazin-2-yl)-2R,6S-dimethyl-piperazin-1-yl]-pyrimidin-2-yl}-ethanol;

(4-{4-[2-(1R-hydroxy-ethyl)-pyrimidin-4-yl]-2R,5S-dimethyl-piperazin-1-yl}-pyrimidin-2-yl)-ethanol; 1S-(4-{4-[2-(1R-hydroxy-ethyl)-pyrimidin-4-yl]-2R,6S-dimethyl-piperazin-1-yl}-pyrimidin-2-yl)-ethanol; prodrugs thereof and pharmaceutically acceptable salts of said compounds and said prodrugs.

Particularly preferred sorbitol dehydrogenase inhibitors for use in the pharmaceutical compositions, methods and kits of this invention include:

1R-{4-[4-(2,6-dimethyl-pyrimidin-4-yl)-2R,6S-dimethyl-piperazin-1-yl]-pyrimidin-2-yl}-ethanol; 1R-(4-{4-[2-(1R-hydroxy-ethyl)-pyrimidin-4-yl]-2R,6S-dimethyl-piperazin-1-yl}-pyrimidin-2-yl)-ethanol; 1R-{4-[4-(2-hydroxymethyl-6-methyl-pyrimidin-4-yl)-2R,6S-dimethyl-piperazin-1-yl]-pyrimidin-2-yl}-ethanol; 1R-(4-{4-[2-(1-S-hydroxy-ethyl)-pyrimidin-4-yl]-2R,6S-dimethyl-piperazin-1-yl}-pyrimidin-2-yl)-ethanol; 1S-(4-{4-[2-(1R-hydroxy-ethyl)-pyrimidin-4-yl]-2R,6S-dimethyl-piperazin-1-yl}-pyrimidin-2-yl)-ethanol; 1-{4-[4-(2-acetyl-pyrimidin-4-yl)-2R,6S-dimethyl-piperazin-1-yl]-pyrimidin-2-yl}-ethanone; 1RS-(4-{4-[2-(1RS-hydroxy-ethyl)-pyrimidin-4-yl]-2R,6S-dimethyl-piperazin-1-yl}-pyrimidin-2-yl)-ethanol; (4-{4-[2-(1R-hydroxy-ethyl)-pyrimidin-4-yl]-3R,5S-dimethyl-piperazin-1-yl}-pyrimidin-2-yl)-ethanone; 1R-{4-[2R,6S-dimethyl-4-(2-morpholin-4-yl-pyrimidin-4-yl)-piperazin-1-yl]-pyrimidin-2-yl}-ethanol; 1R-(4-{2R,6S-dimethyl-4-[2-(4-methyl-piperazin-1-yl)-pyrimidin-4-yl]-piperazin-1-yl}-pyrimidin-2-yl)-ethanol; 1R-{4-[2R,6S-dimethyl-4-(2-[1,2,4]triazol-1-yl-pyrimidin-4-yl)-piperazin-1-yl]-pyrimidin-2-yl}-ethanol; 1R-(4-{4-[2-(1R-hydroxy-ethyl)-pyrimidin-4-yl]-2R,6R-dimethyl-piperazin-1-yl}-pyrimidin-2-yl)-ethanol; 1R-(4-{4-[2-(4-ethyl-piperazin-1-yl)-pyrimidin-4-yl]-2R,6S-dimethyl-piperazin-1-yl}-pyrimidin-2-yl)-ethanol; 1R-(4-{2R,6S-dimethyl-4-[2-(4-methyl-imidazol-1-yl)-pyrimidin-4-yl]-piperazin-1-yl}-pyrimidin-2-yl)-ethanol; 1R-{4-[4-(4-cyclopropyl-[1,3,5]triazin-2-yl)-2R,6S-dimethyl-piperazin-1-yl]-pyrimidin-2-yl}-ethanol; 1R-{4-[4-(4,6-dimethyl-[1,3,5]triazin-2-yl)-2R,6S-dimethyl-piperazin-1-yl]-pyrimidin-2-yl}-ethanol; 1R-{4-[4-(4-hydroxymethyl-6-phenyl-[1,3,5]triazin-2-yl)-2R,6S-dimethyl-piperazin-1-yl]-pyrimidin-2-yl}-ethanol; 1R-{4-[4-(4-methoxy-6-methoxymethyl-[1,3,5]triazin-2-yl)-2R,6S-dimethyl-piperazin-1-yl]-pyrimidin-2-yl}-ethanol; 1R-{4-[2R,6S-dimethyl-4-(4-methyl-[1,3,5]triazin-2-yl)-piperazin-1-yl]-pyrimidin-2-yl-ethanol; and 1R-(4-{4-[2-(2,4-dimethyl-imidazol-1-yl)-pyrimidin-4-yl]-2R,6S-dimethyl-piperazin-1-yl}-pyrimidin-2-yl)-ethanol; prodrugs thereof and pharmaceutically acceptable salts of said compounds and said prodrugs.

Preferred statins for use in the pharmaceutical compositions, methods and kits of this invention include: atorvastatin, simvastatin, pravastatin, rivastatin, mevastatin, fluindostatin, velostatin, fluvastatin, dalvastatin, dihydrocompactin, compactin, lovastatin; prodrugs thereof and pharmaceutically acceptable salts of said statins and said prodrugs.

Particularly preferred statins for use in the pharmaceutical compositions, methods and kits of this invention include atorvastatin, rivastatin, simvastatin, pravastatin, mevastatin, lovastatin, prodrugs thereof and pharmaceutically acceptable salts of said statins and said prodrugs.

Even more particularly preferred statins for use in the pharmaceutical compositions, methods and kits of this invention include atorvastatin and atorvastatin calcium.

This invention is also directed to kits for achieving a therapeutic effect in a mammal comprising:

a. an amount of a statin, a prodrug thereof or a pharmaceutically acceptable salt of said statin or said prodrug and a pharmaceutically acceptable vehicle, carrier or diluent in a first unit dosage form;

b. an amount of a sorbitol dehydrogenase inhibitor, a prodrug thereof or a pharmaceutically acceptable salt of said sorbitol dehydrogenase inhibitor or said prodrug and a pharmaceutically acceptable vehicle, carrier or diluent in a second unit dosage form; and

c. a container.

This invention is also directed to methods for treating a mammal in need of therapeutic treatment comprising administering to said mammal

(a) an amount of a first compound, said first compound being a statin, a prodrug thereof or a pharmaceutically acceptable salt of said statin or said prodrug; and

(b) an amount of a second compound, said second compound being a sorbitol dehydrogenase inhibitor, a prodrug thereof or a pharmaceutically acceptable salt of said sorbitol dehydrogenase inhibitor or said prodrug;

wherein said first compound and said second compound are each optionally and independently administered together with a pharmaceutically acceptable vehicle, carrier or diluent. The methods of this invention include therapeutic treatment of diabetic complications as well as therapeutic treatment of atherosclerosis. Diabetic complications which may be treated by the methods of this invention include, inter alia, diabetic neuropathy, diabetic nephropathy, diabetic cardiomyopathy, diabetic retinopathy, diabetic microangiopathy, diabetic macroangiopathy, cataracts and foot ulcers. Humans are especially preferred mammals which are treated in the methods of this invention.

### DETAILED DESCRIPTION OF THE INVENTION

The combinations of this invention comprise two active components: a statin, a prodrug thereof or a pharmaceutically acceptable salt of said statin or said prodrug and a sorbitol dehydrogenase inhibitor, a prodrug thereof or a pharmaceutically acceptable salt of said sorbitol dehydrogenase inhibitor or said prodrug. The combination of this invention may also include a pharmaceutically acceptable vehicle, carrier or diluent.

The first component of the combinations of this invention is a statin, a prodrug thereof or a pharmaceutically acceptable salt of said statin or said prodrug. The term “statin”, where used in the description and the appendant claims, is synonymous with the terms “3-hydroxy-3-methylglutaryl-Coenzyme A reductase inhibitor” and “HMG-CoA reductase inhibitor.” These three terms are used interchangeably throughout the description and appendant claims. As the synonyms suggest, statins are inhibitors of 3-hydroxy-3-methylglutaryl-Coenzyme A reductase and as such are effective in lowering the level of blood plasma cholesterol. Statins and pharmaceutically acceptable salts thereof are particularly useful in lowering low density lipoprotein cholesterol (LDL-C) levels in mammals and particularly in humans.

The HMG-CoA reductase inhibitors suitable for use herein include, but are not limited to, atorvastatin, simvastatin, pravastatin, rivastatin, mevastatin, fluindostatin, velostatin, fluvastatin, dalvastatin, dihydrocompactin, compactin or lovastatin; a prodrug of said statin or a pharmaceutically acceptable salt of said statin or said prodrug.

The statins disclosed herein are prepared by methods well known to those skilled in the art. Specifically, atorvastatin may be prepared according to the method disclosed in U.S. Pat. No. 4,681,893. Atorvastatin calcium may be prepared according to the method disclosed in U.S. Pat. No. 5,273,995. Simvastatin may be prepared according to the method disclosed in U.S. Pat. No. 4,444,784. Pravastatin may be prepared according to the method disclosed in U.S. Pat. No. 4,346,227. Cerivastatin may be prepared according to the method disclosed in U.S. Pat. No. 5,502,199. Cerivastatin may alternatively be prepared according to the method disclosed in European Patent Application Publication No. EP617019. Mevastatin may be prepared according to the method disclosed in U.S. Pat. No. 3,983,140. Velostatin may be prepared according to the methods disclosed in U.S. Pat. No. 4,448,784 and U.S. Pat. No. 4,450,171. Fluvastatin may be prepared according to the method disclosed in U.S. Pat. No. 4,739,073. Compactin may be prepared according to the method disclosed in U.S. Pat. No. 4,804,770. Lovastatin may be prepared according to the method disclosed in U.S. Pat. No. 4,231,938. Dalvastatin may be prepared according to the method disclosed in European Patent Application Publication No. 738510 A2. Fluindostatin may be prepared according to the method disclosed in European Patent Application Publication No. 363934 A1. Dihydrocompactin may be prepared according to the method disclosed in U.S. Pat. No. 4,450,171. Each of the above is incorporated herein by reference.

It will be recognized that certain of the statins and sorbitol dehydrogenase inhibitors used in the pharmaceutical compositions, methods and kits of this invention contain either a free carboxylic acid or a free amine group as part of the chemical structure. Further, certain statins within the scope of this invention contain lactone moieties, which exist in equilibrium with the free carboxylic acid form. These lactones can be maintained as carboxylates by preparing pharmaceutically acceptable salts of the lactone. Thus, this invention includes pharmaceutically acceptable salts of those carboxylic acids or amine groups. The expression “pharmaceutically acceptable salts” includes both pharmaceutically acceptable acid addition salts and pharmaceutically acceptable cationic salts. The expression “pharmaceutically-acceptable cationic salts” is intended to define but is not limited to such salts as the alkali metal salts, (e.g., sodium and potassium), alkaline earth metal salts (e.g., calcium and magnesium), aluminum salts, ammonium salts, and salts with organic amines such as benzathine (N,N′-dibenzylethylenediamine), choline, diethanolamine, ethylenediamine, meglumine (N-methylglucamine), benethamine (N-benzylphenethylamine), diethylamine, piperazine, tromethamine (2-amino-2-hydroxymethyl-1,3-propanediol) and procaine. The expression “pharmaceutically-acceptable acid addition salts” is intended to define but is not limited to such salts as the hydrochloride, hydrobromide, sulfate, hydrogen sulfate, phosphate, hydrogen phosphate, dihydrogenphosphate, acetate, succinate, citrate, methanesulfonate (mesylate) and p-toluenesulfonate (tosylate) salts.

The pharmaceutically-acceptable cationic salts of statins or sorbitol dehydrogenase inhibitors containing free carboxylic acids may be readily prepared by reacting the free acid form of the statin or sorbitol dehydrogenase inhibitor with an appropriate base, usually one equivalent, in a co-solvent. Typical bases are sodium hydroxide, sodium methoxide, sodium ethoxide, sodium hydride, potassium methoxide, magnesium hydroxide, calcium hydroxide, benzathine, choline, diethanolamine, piperazine and tromethamine. The salt is isolated by concentration to dryness or by addition of a non-solvent. In many cases, salts are preferably prepared by mixing a solution of the acid with a solution of a different salt of the cation (e.g., sodium or potassium ethylhexanoate, magnesium oleate), employing a solvent (e.g., ethyl acetate) from which the desired cationic salt precipitates, or can be otherwise isolated by concentration and/or addition of a non-solvent.

The pharmaceutically acceptable acid addition salts of statins or sorbitol dehydrogenase inhibitors containing free amine groups may be readily prepared by reacting the free base form of the statin or sorbitol dehydrogenase inhibitor with the appropriate acid. When the salt is of a monobasic acid (e.g., the hydrochloride, the hydrobromide, the p-toluenesulfonate, the acetate), the hydrogen form of a dibasic acid (e.g., the hydrogen sulfate, the succinate) or the dihydrogen form of a tribasic acid (e.g., the dihydrogen phosphate, the citrate), at least one molar equivalent and usually a molar excess of the acid is employed. However, when such salts as the sulfate, the hemisuccinate, the hydrogen phosphate or the phosphate are desired, the appropriate and exact chemical equivalents of acid will generally be used. The free base and the acid are usually combined in a co-solvent from which the desired salt precipitates, or can be otherwise isolated by concentration and/or addition of a non-solvent.

The expression “prodrug” refers to compounds that are drug precursors which, following administration, release the drug in vivo via some chemical or physiological process (e.g., a prodrug on being brought to the physiological pH or through enzyme action is converted to the desired drug form). Both the statins and the sorbitol dehydrogenase inhibitors used in the combinations, pharmaceutical compositions, methods and kits of this invention may be prepared as prodrugs. The preparation of prodrugs is straightforward and may be achieved using methods well known to those skilled in the art. All such prodrugs are within the scope of the combinations, pharmaceutical compositions, methods and kits of this invention.

In addition, the statins, prodrugs thereof and pharmaceutically acceptable salts of said statins and said prodrugs used in the combinations of the instant invention may occur as hydrates or solvates. Further, the sorbitol dehydrogenase inhibitors, prodrugs thereof and pharmaceutically acceptable salts of said sorbitol dehydrogenase inhibitors and said prodrugs used in the combinations of the instant invention may also occur as hydrates or solvates. Said hydrates and solvates are also within the scope of the invention.

Any sorbitol dehydrogenase inhibitor may be used as one of the active ingredients in the combinations, pharmaceutical compositions, methods and kits of the instant invention. Compounds of the Formula A, above, may be prepared as set forth in U.S. Pat. No. 5,728,704 and/or U.S. Pat. No. 5,866,578.

In general the sorbitol dehydrogenase inhibitors of Formula I of this invention can be made by processes which include processes known in the chemical arts, particularly in light of the description contained herein. Certain processes for the manufacture of the sorbitol dehydrogenase inhibitors of formula I of this invention are provided as further features of the invention and are illustrated by the following reaction schemes.

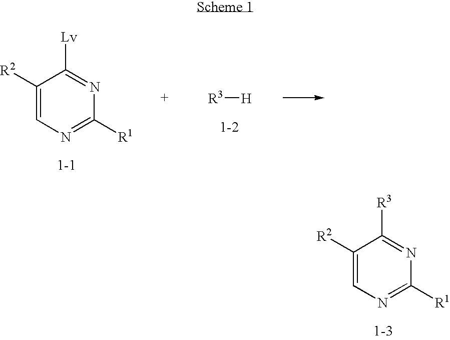

Compounds of formula 1-3 (i.e., formula I) are prepared as set forth in Scheme 1, particularly as described below.

Compounds of formula 1-3 are prepared by the displacement reaction of a pyrimidine of the formula 1-1 where R¹ and R² are defined herein. Lv is a leaving group preferably selected from fluoro, chloro, bromo, iodo, thiomethyl, methylsulfone, or OSO₂J wherein J is (C₁-C₆)-lower alkyl, trifluoromethyl, pentafluoroethyl, phenyl optionally substituted with up to three (C₁-C₄)alkyl, nitro or halo. The leaving group Lv is displaced by an amine of the formula 1-2 where R³ is defined above. The reaction is conducted in the presence of a non-aqueous base, preferably an organic amine or an inorganic base. Preferred organic amines include triethylamine, pyridine, dimethylaminopyridine and N,N′-diisopropylethylamine (Hunig's base). Preferred inorganic bases include alkaline metal carbonates and bicarbonates such as sodium or potassium carbonate and sodium or potassium bicarbonate. An especially preferred inorganic base is potassium carbonate. An especially preferred organic amine is triethylamine. Alternatively, an excess of the reacting amine 1-2 can be used as the base for this reaction. The reaction can be conducted in the absence of solvent or in a reaction inert solvent. Where used herein, “reaction inert solvent” refers to a solvent which does not interact with starting materials, reagents, intermediates or products in a manner which adversely affects the yield of the desired product. Preferred reaction inert solvents include aqueous media, pyridine, (C₁-C₄)alcohol, (C₂-C₆)glycol, halocarbon, aliphatic/aromatic hydrocarbon, ethereal solvent, polar aprotic solvent, ketonic solvent, or combinations thereof. The reaction time ranges from 15 minutes to 3 days and the reaction temperature ranges from 0° C. to 180° C. Conveniently, the reaction may be conducted at the reflux temperature of the solvent being used. The reaction is preferably conducted at ambient pressure. The term ambient pressure, where used herein, refers to the pressure of the room in which the reaction is being conducted. The term ambient temperature, where used herein, refers to the temperature of the room in which the reaction is being conducted.

When R¹ contains a hydroxy group, the hydroxyl group may or may not be protected. When the hydroxyl group is protected, the protecting group may be any suitable hydroxyl protecting group. The conditions used to remove such optional hydroxyl protecting groups contained in R¹ in compounds of formula 1-3 are as follows. When the protecting group is an ester, removal of such ester protecting groups is conducted under basic conditions using inorganic hydroxides or carbonates, preferably lithium hydroxide, sodium hydroxide, potassium hydroxide or potassium carbonate. The reaction is carried out in a reaction inert solvent, preferably an alcoholic solvent. Especially preferred is methanol or methanol in combination with co-solvents such as water, tetrahydrofuran, or dioxane. The reaction time ranges from 15 minutes to 24 hours and the reaction temperature ranges from 0° C. to 100° C. or to the reflux temperature of the solvent(s) of use. Alternatively, ester cleavage may be accomplished under acidic conditions. It is preferred to utilize aqueous hydrochloric acid, generally 2 N to concentrated, with or without a co-solvent. When a co-solvent is used, dioxane or methanol are preferred. The reaction time ranges from 4 hours to 3 days and the reaction temperature ranges from 0° C. to 60° C.

When the protecting group is an alkyl ether, removal of such alkyl ether protecting groups is conducted using well known dealkylative conditions. For example, the alkyl ether may be cleaved by reaction with boron tribromide or diethylboron bromide in a reaction inert solvent, preferably a halocarbon solvent. It will be recognized by those skilled in the art that a buffer such as triethylamine may facilitate the reaction. The reaction times range from 15 minutes to 24 hours and the reaction temperature ranges from 0° C. to 60° C. In addition, a benzyl ether protecting group can be removed via standard or transfer hydrogenolysis using a palladium catalyst such as palladium on carbon. The hydrogenolysis reaction is conducted under a hydrogen atmosphere at ambient pressure to 50 psi in a reaction inert solvent, preferably methanol. The hydrogen source may be hydrogen gas, ammonium formate or trialkylammonium formate or cyclohexene. The reaction temperature ranges from room temperature to the reflux temperature of the solvent employed. The reaction time ranges from 15 minutes to 24 hours.

When a silyl ether protecting group is employed, removal of such silyl ether protecting groups is conducted under acidic conditions, preferably with aqueous hydrochloric acid such as 1 N to 6 N hydrochloric acid. The de-protection may be carried out in the presence of a co-solvent such as methanol or tetrahydrofuran. The reaction time ranges from 2 hours to 48 hours and the reaction temperature ranges from 0° C. to 100° C. Alternatively, the silyl ether protecting group may be removed via fluoride-mediated deprotection. In this case, deprotection is conducted using tetrabutylammonium fluoride or one of a variety of hydrofluoric acid sources in a reaction inert solvent. It is preferred to use ethereal solvents such as diethyl ether, dioxane or tetrahydrofuran, with tetrahydrofuran being especially preferred. The reaction time ranges from 2 hours to 48 hours and the reaction temperatures range from 0° C. to the reflux temperature of the solvent being used. Other methods for removal of the aforementioned protecting groups are well known to those skilled in the art or can be found in Greene, T. W.; Wuts, P. G. M., Protective Groups in Organic Synthesis, 2^{nd} ed.; John Wiley and Sons Inc.: New York, 1991. Other suitable hydroxyl protecting groups and methods for their removal may be found also be found therein. The method of Scheme I is preferred when R³ is R^{3k, I, m, n, o, p and q}. Thus, compounds of formula 1-2 are reacted with compounds of formula 1-1. Compounds of formula 1-2 where R³ is R^{3k, I, m, n, o, p or q} are commercially available or can be prepared by methods well known to those skilled in the art.

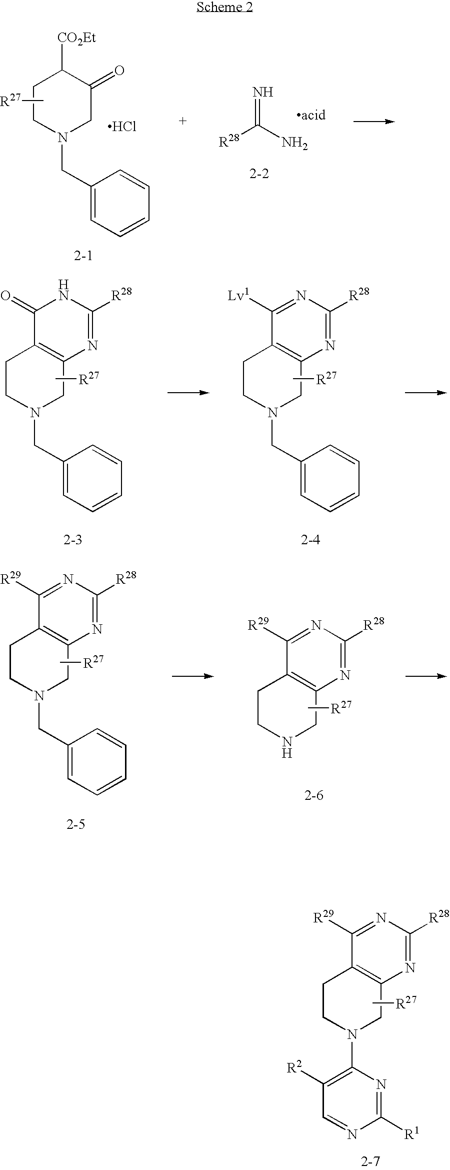

Compounds of formula 2-7 are prepared as set forth in Scheme 2, particularly as described below.

Where R²⁷ is H, ethyl 1-benzyl-3-oxo-4-piperidine-carboxylate hydrochloride, the compound of formula 2-1, which is available from Aldrich, is condensed with compounds of formula 2-2 to give compounds of formula 2-3. The compounds of formula 2-1 where R²⁷ is not H can be prepared according to methods well known to those skilled in the art. The reaction is conducted in the presence of excess base including non-aqueous bases, organic amines and inorganic bases. Preferred organic amines include triethylamine and pyridine. Preferred non-aqueous bases include alkaline metal (C₁-C₄)alkoxides. Preferred inorganic bases include potassium carbonate. The reaction is conducted in a reaction inert solvent. Preferred such solvents include (C₁-C₄)alcohols, aromatic or aliphatic hydrocarbons, polar aprotic solvents, halocarbons, and ethereal solvents. (C₁-C₄)Alcohols are especially preferred. The reaction time ranges from 2 hours to 3 days. The reaction temperature ranges from ambient temperature to the reflux temperature of the solvent being employed. The reaction is preferably run at ambient pressure but may be conducted at pressures up to 250 psi.

Compounds of formula 2-4 are prepared from compounds of formula 2-3 by converting a compound of formula 2-3 into an activated compound of formula 2-4 where Lv¹ is selected from fluoro, chloro, bromo, iodo, trifluoromethanesulfonate, (C₁-C₆)alkylsulfonate, or phenylsulfonate, wherein said phenyl is optionally substituted with up to three (C₁-C₄)alkyl, halo or nitro. This reaction is accomplished by reacting compounds of formula 2-3 with a chlorinating agent such as phosphorus oxychloride and/or phosphorus pentachloride to provide compounds of formula 2-4 where Lv¹ is chloro. This reaction is conducted at ambient pressure in the absence of solvent or in a reaction inert solvent, preferably a halocarbon solvent at temperatures ranging from ambient temperature to 180° C. Treatment of the chloro compound thus formed with the requisite mineral acid provides a compound of formula 2-4 where Lv¹ is bromo or iodo. A sulfonate of formula 2-4 is prepared by reaction of a compound of formula 2-3 with a sulfonic acid chloride or anhydride in the presence of an organic amine base, preferably triethylamine or pyridine. In certain cases recognized by those skilled in the art, it may be necessary to add a catalyst to the reaction. In those cases, a preferred catalyst is 4-dimethylaminopyridine. This reaction is conducted at ambient pressure in a reaction inert solvent, preferably pyridine, a halocarbon such as chloroform, dichloromethane or carbon tetrachloride, an aromatic or aliphatic hydrocarbon, an ethereal solvent, or combinations thereof. The reaction temperature ranges from −20° C. to 100° C. and the reaction time ranges from 15 minutes to 1 day.

Compounds of formula 2-5 wherein R²⁹ is defined above are prepared from compounds of formula 2-4 by a reduction reaction or by displacement of Lv¹ with a nucleophile. The reduction is conducted with a reducing agent, preferably ammonium formate or hydrogen gas, in a reaction inert solvent. The reduction is conducted in the presence of a palladium catalyst at ambient pressure or under a hydrogen pressure of up to 50 psi. Preferred solvents include (C₁-C₄)alcohols such as methanol and ethanol, and ether solvents such as diethyl ether, dioxane and tetrahydrofuran. The nucleophilic displacement reaction may be conducted by adding the nucleophile directly or by pre-forming the nucleophile separately or in situ from a nucleophile precursor. Preferred nucleophiles include organoaluminum, organoboron, organocopper, organotin, organozinc or Grignard reagent; R²⁹—H; or, where R²⁹ contains a hydroxyl or thiol group, the anion of R²⁹. The term “organo” in the terms organoaluminum, organoboron, organocopper, organotin and organozinc refers to an organic radical selected from R²⁹. It will be recognized by those skilled in the art that transition-metal catalysts may be required to effect reaction in certain displacement reactions. When required, such transition metal catalysts may include palladium(0), palladium(II), nickel(0), and nickel(II) complexes. Palladium(II) bis(diphenylphosphinobutane) dichloride is a preferred such catalyst. Additionally, an aqueous or non-aqueous base may be required in the displacement reaction. Preferred such bases include sodium carbonate, sodium hydride, triethylamine and sodium tert-butoxide. The reaction is conducted at ambient pressure in a reaction inert solvent such as a halocarbon, an aromatic or aliphatic hydrocarbon, an ether or a polar aprotic solvent or a combination thereof. In certain cases, a (C₁-C₄)alcohol is used as a solvent or co-solvent. The reaction temperature ranges from −20° C. to the reflux temperature of the solvent employed. The reaction time ranges from 1 hour to 3 days.

Compounds of formula 2-6 are prepared by removal of the benzyl protecting group from compounds of formula 2-3 or 2-5. This transformation is accomplished using the freebase, or preferably the pre-formed hydrochloride or similar salt, under standard or transfer hydrogenolysis conditions. The catalysts which may be used in the hydrogenolysis reaction include, but are not limited to, palladium on carbon, palladium hydroxide on carbon and platinum(IV) oxide. The reaction is conducted in a reaction inert solvent, preferably methanol or ethanol and the reaction temperature ranges from room temperature to the reflux temperature of the solvent being employed. The hydrogen source is hydrogen gas, ammonium formate, trialkylammonium formate, or cyclohexene. The reaction time ranges from 15 minutes to 3 days. Generally the reaction is conducted at ambient pressure but pressures of up to 50 psi of hydrogen may be employed. Alternatively, if appropriate, the benzyl protecting group is removed in two steps via chloroformate-induced acylative dealkylation. This involves reaction with a chloroformate derivative to form a carbamate followed by cleavage of the carbamate. While this reaction is preferably conducted with 1-chloroethyl chloroformate and sodium iodide catalysis, it will be recognized by those skilled in the art that catalysis may not be required in certain cases. The reaction is conducted at ambient temperature in a reaction inert solvent such as a halocarbon, an aromatic or aliphatic hydrocarbon, a ketone, an ether or a polar aprotic solvent. The reaction temperature ranges from −78° C. to the reflux temperature of the solvent being employed and the reaction time ranges from 15 minutes to 1 day. Cleavage of the carbamate formed by reaction with 1-chloroethyl chloroformate is accomplished upon exposure to methanol or ethanol at ambient pressure to give compounds of formula 2-6 as a hydrochloride salt. The reaction proceeds at temperatures from room temperature to the reflux temperature of the solvent being employed and the reaction time ranges from 15 minutes to 1 day. Deprotection conditions for other carbamates can be found in Greene, T. W.; Wuts, P. G. M. Protective Groups in Organic Synthesis, 2^{nd} ed.; John Wiley and Sons Inc.: New York, 1991, pp 315-348.

Compounds of formula 2-7 are prepared from the displacement reaction of amine 2-6 as described in Scheme 1, where the amine 2-6 is equivalent to R³—H.

Alternatively, compounds of formula 2-7 where R²⁹ is as defined above are prepared from compounds of formula 2-3 wherein R²⁹ is OH according to the sequence outlined in Scheme 2a below, wherein the conditions are as set forth as described for Scheme 2.

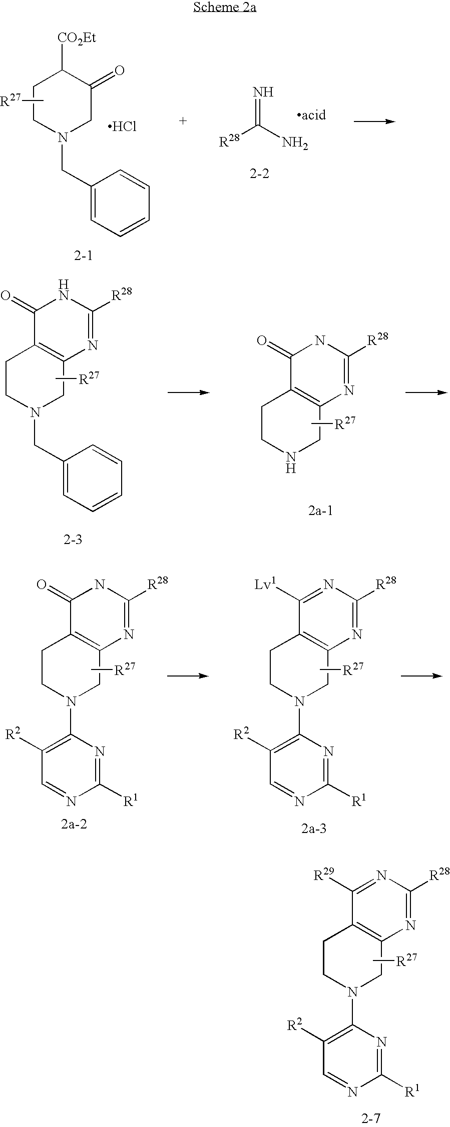

Compounds of formula 2-2 which are used in Schemes 2 and 2a above are commercially available or are prepared according to methods well known to those skilled in the art, such as those described in March, J. Advanced Organic Chemistry, 3^{rd} ed.; John Wiley and Sons.: New York, 1985, p 359, 374.

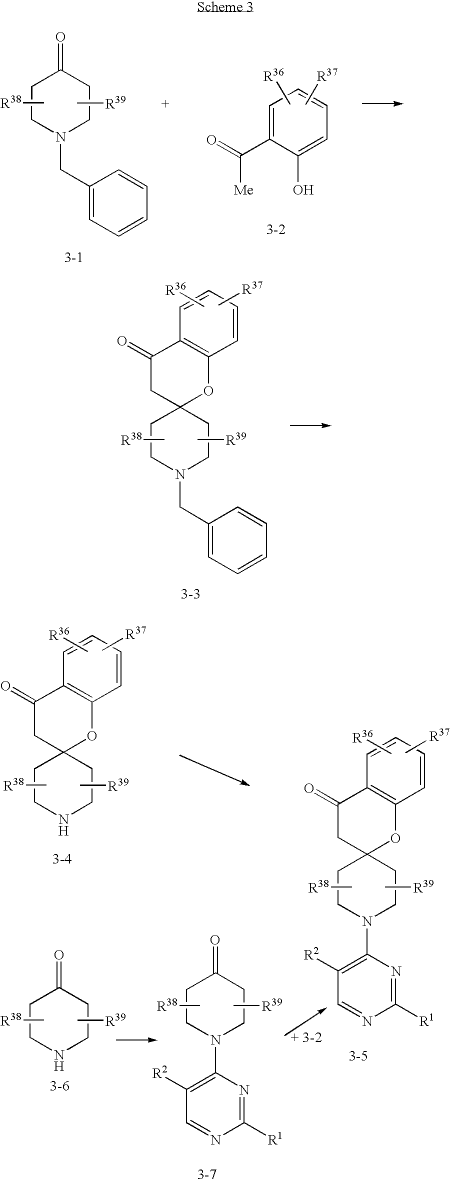

Compounds of formula 3-5 are prepared as set forth in Scheme 3 above and more particularly as described below.

Compounds of formula 3-3 are prepared by condensing a compound of formula 3-1 with a compound of formula 3-2. Where R³⁸ and R³⁹ are each H, the compound of formula 3-1 is 1-benzyl-4-piperidone, which is commercially available from Aldrich. Compounds of formula 3-2 are either commercially available or can be prepared according to methods well known to those skilled in the art, particularly according to methods set forth in March, J. Advanced Organic Chemistry, 3^{rd} ed.; John Wiley and Sons Inc.: New York, 1985, pp 499-500. The reaction is conducted at ambient pressure in the presence of a secondary amine. Generally an excess of the secondary amine, preferably pyrrolidine, piperidine, morpholine or diethylamine, is used. An especially preferred secondary amine is pyrrolidine. The reaction is conducted in a reaction inert solvent, preferably a (C₁-C₄)alcohol, an aromatic or aliphatic hydrocarbon, a polar aprotic solvent, a halocarbon or an ether. An especially preferred solvent is ethanol. The reaction time ranges from 2 hours to 3 days and the reaction temperature ranges from ambient temperature to the reflux temperature of the solvent being employed.

Compounds of the formula 3-4 are prepared by removal of the benzyl protecting group from compounds of formula 3-3. This transformation is conducted in a manner analogous to the procedure set forth for the preparation of compounds of formula 2-6 above.

Compounds of formula 3-5 are prepared from the displacement reaction of amine 3-4 as described in Scheme 1, where the amine 3-4 is equivalent to R³—H.

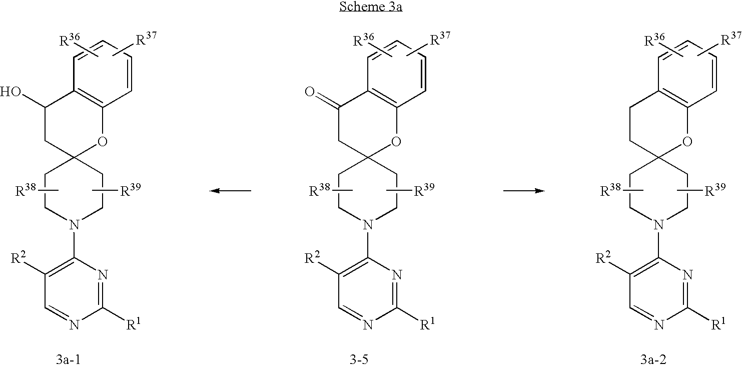

Compounds of formulas 3a-1 and 3a-2 are prepared as shown in Scheme 3a from compounds of formula 3-5. Thus, to prepare a compound of 3a-1, a compound of formula 3-5 is reduced with a common reducing agent, such as, for example, sodium borohydride, lithium aluminum hydride or diisobutylaluminum hydride. Other reducing agents capable of effecting the reduction of a ketone to an alcohol are well known to those skilled in the art (e.g., Larock, R. D. Comprehensive Organic Transformations, VCH Publishers, Inc.: New York, 1989, pp 527-547). Likewise, compounds of formula 3a-2 are prepared from compounds of formula 3-5 by reduction with reducing agents capable of reducing a ketone completely to a methylene group. A preferred such reducing agent is aluminum trichloride/borane-tert-butylamine complex. Other such reducing agents are well known to those skilled in the art (e.g., J. Org. Chem. 1989, 54, 4350; Larock, R. D. Comprehensive Organic Transformations, VCH Publishers, Inc.: New York, 1989, pp 35-37). It will be recognized by those skilled in the art that the transformation of 3-5 to 3a-1 or 3a-2 can be conducted at different points in Scheme 3, depending upon the dynamics of the particular system.

Alternatively, compounds of formula 3-5 wherein R³⁸ and R³⁹ are hydrogen can be prepared from 4-piperidone monohydrate monochloride in a manner analogous to the procedure described in Scheme 1, where the amine 3-6 is equivalent to R³—H to give compounds of formula 3-7. Compounds of formula 3-7 can be reacted with compounds of formula 3-2 in a manner analogous to the procedure set forth for the synthesis of compounds of formula 3-3 to afford compounds of formula 3-5.

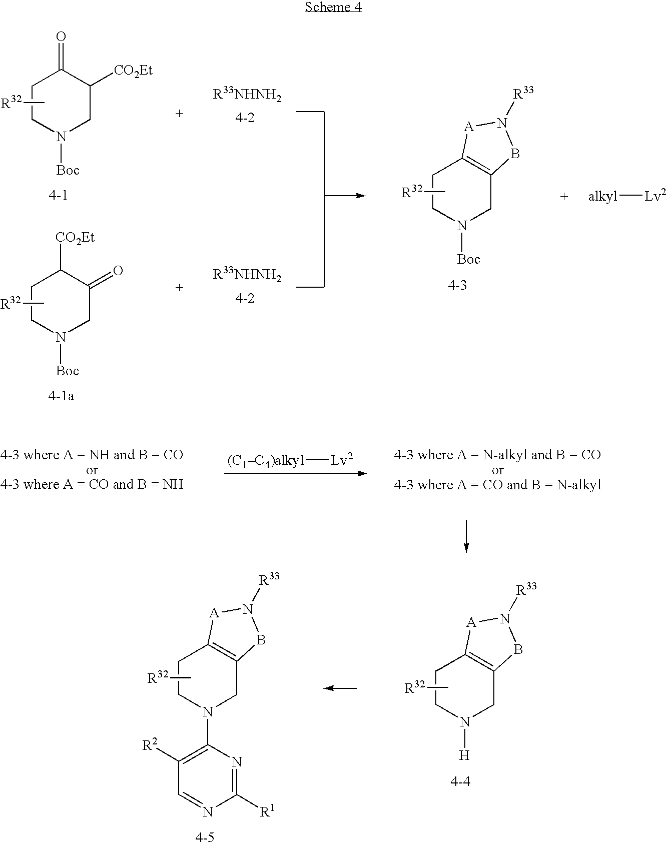

Compounds of formula 4-5 are prepared according to Scheme 4 and more particularly as described below.

Compounds of formula 4-3 are prepared by reacting a compound of formula 4-2 with a compound of formula 4-1 or 4-1a. Compounds of formula 4-1 and 4-1a are prepared according to methods well known to those skilled in the art. Where R³² is hydrogen, 4-oxo-piperidine-1,3-dicarboxylic acid 1-tert-butyl ester 3-ethyl ester is condensed with a compound of formula 4-2 to afford a compound of formula 4-3. Said compounds of formula 4-2 are readily available from well known commercial vendors, known in the literature, or are synthesized under standard conditions well known to those skilled in the art. Preferred conditions to prepare compounds of formula 4-3 from a compound of formula 4-1 where A is CO and B is NH or from a compound of formula 4-1a where A is NH and B is CO can be found in March, J. Advanced Organic Chemistry, 3^{rd} ed.; John Wiley and Sons Inc.: New York, 1985, p 1163. The reaction is conducted at ambient pressure in a reaction inert solvent. Preferred such solvents include aqueous media, a (C₁-C₄)alcohol, glacial acetic acid, an aromatic or aliphatic hydrocarbon, a polar aprotic solvent, a halocarbon and ethers or combinations thereof. The reaction time ranges from 2 hours to 3 days and the reaction temperature ranges from ambient temperature to the reflux temperature of the solvent being used. An optional second step using aqueous or non-aqueous base may be employed in certain cases which will be recognized by those skilled in the art. This second step is conducted at ambient pressure in a reaction inert solvent. Preferred such solvents include aqueous media, a (C₁-C₄)alcohol, glacial acetic acid, an aromatic or aliphatic hydrocarbon, a polar aprotic solvent, a halocarbon and ethers or combinations thereof. The reaction time ranges from 2 hours to 3 days and the reaction temperature ranges from ambient temperature to the reflux temperature of the solvent being used.

Compounds of formula 4-3 wherein B is CO and A is N-alkyl or wherein B is N-alkyl and A is CO are prepared by alkylation of compounds of formula 4-3 where B is CO and A is NH or wherein B is NH and A is CO, respectively. The anion of those compounds of formula 4-3 is formed by reaction with an appropriate base. Preferred such bases include sodium hydride and sodium hexamethyldisilazide, although other bases may be used where conditions warrant, as determined by the skilled person. The reaction is conducted in a reaction inert solvent, preferably an ether such as tetrahydrofuran, diethyl ether, dioxane or diglyme or polar aprotic solvent such as dimethylformamide. The reaction proceeds at ambient pressure and at temperatures ranging from −100° C. to ambient temperature. The reaction times are from 10 minutes to 2 hours. Addition of (C₁-C₄)alkyl halides or (C₁-C₄)alkylsulfonates such as mesylate, tosylate or nosylate to the anion of 4-3 proceeds at ambient pressure and at temperatures ranging from −20° C. to 50° C. The reaction times range from 10 minutes to 1 day.

Compounds of formula 4-4 are prepared form compounds of formula 4-3 wherein A is N-alkyl and B is CO or A is CO and B is N-alkyl via acid-catalyzed deprotection of the Boc carbamate under standard conditions, for example, hydrochloric acid or trifluoroacetic acid in a reaction inert solvent or in the absence of solvent. Such conditions are known to those skilled in the art. Exemplary conditions are disclosed in Greene, T. W.; Wuts, P. G. M. Protective Groups in Organic Synthesis, 2^{nd} ed.; John Wiley and Sons Inc.: New York, 1991, pp 327-330.

Compounds of formula 4-5 are prepared by the displacement reaction of amine 4-4 as described in Scheme 1, where the amine 4-4 is equivalent to R³—H.

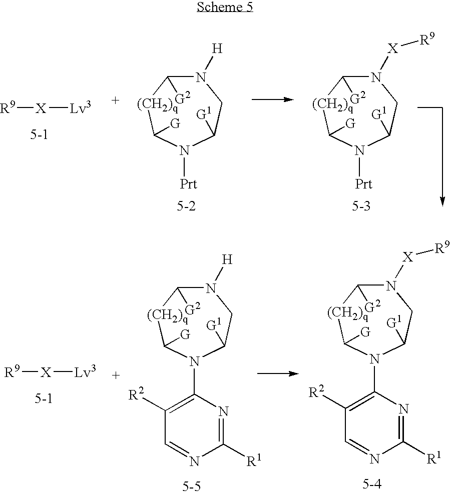

Compounds of formula 5-4 where X is a covalent bond and G, G¹, G², q, R¹, R², R⁶, R⁷ and R⁸ are as defined above are prepared according to Scheme 5 above and particularly as described below.

Compounds of formula 5-3 are prepared by reaction of a compound of formula 5-1 with a compound of formula 5-2 where Prt is an optional amine protecting group selected from benzyl and C₂R⁹⁰, where R⁹⁰ is selected from (C₁-C₄)alkyl, (C₁-C₄)allyl, trichloroethyl and benzyl substituted by up to two (C₁-C₄)alkoxy. Compounds of formula 5-1 where R⁹ is Ar¹ and Lv³ is halo, (C₁-C₄)alkylsulfide, (C₁-C₄)alkylsulfone, trifluoromethanesulfonate, (C₁-C₆)alkylsulfonate or phenylsulfonate, where said phenyl is optionally substituted with up to three halo, nitro or (C₁-C₄)alkyl are commercially available or are readily prepared according to methods well known to those skilled in the art. For example, to prepare compounds of formula 5-1 wherein Lv³ is chloro, a compound of formula Ar¹—OH, or the Ar¹—(═O) tautomer thereof, is reacted with a chlorinating agent such as phosphorus oxychloride and/or phosphorus pentachloride. This chlorinating reaction is conducted at ambient pressure in the absence of solvent or in a reaction inert solvent, preferably a halocarbon solvent, at temperatures ranging from ambient temperature to 180° C. Treatment of the chloro compound with the requisite mineral acid provides compounds of formula 5-1 where Lv³ is bromo or iodo. Compounds of formula 5-1 wherein Lv³ is trifluoromethanesulfonate, (C₁-C₆)alkylsulfonate or phenylsulfonate are prepared from a compound of formula Ar¹—OH, or the Ar¹—(═O) tautomer thereof, by reaction with a sulfonic acid chloride or anhydride in the presence of a base, preferably an organic amine such as triethylamine, N,N′-diisopropylethylamine, dimethylaminopyridine or pyridine. In certain cases it will be recognized by those skilled in the art that a catalyst will be required to effect reaction. In those cases, a preferred catalyst is 4-dimethylaminopyridine. This reaction is conducted at ambient pressure in a reaction inert solvent such as pyridine, a halocarbon, an aromatic or aliphatic hydrocarbon, an ether, or a combination thereof. The reaction temperature ranges from −20° C. to 100° C. and the reaction time ranges from 15 minutes to 1 day. Compounds of formula 5-1 where Lv³ is thiomethyl are prepared by reacting a compound of formula Ar¹—SH, or the Ar¹—(═S) tautomer thereof, with methyl iodide or dimethylsulfate in the presence of an inorganic base, preferably potassium carbonate. These reactions are conducted at ambient pressure in a reaction inert solvent, preferably an ether or a polar aprotic solvent. An especially preferred polar aprotic solvent is dimethylformamide at a temperature ranging from 0° C. to 100° C. Compounds of formula 5-1 where Lv³ is methylsulfone are prepared from a compound of formula 5-1 where LV³ is thiomethyl by oxidation thereof according to procedures well known to those skilled in the art, specifically as set forth in March, J. Advanced Organic Chemistry, 3^{rd} ed.; John Wiley and Sons.: New York, 1985, pp 1089-1090.

A representative set of compounds of formula 5-1 which are commercially available or which can be prepared according to methods analogous to a literature procedure include 4-chloropyridine (Aldrich, P.O. Box 355, Milwaukee, Wis. 53201, USA), 3-chloro-6-methyl-pyridazine (Maybridge, c/o Ryan Scientific, 443 Long Point Road, Suite D, Mount Pleasant, S.C. 29464, USA), 2-chloro-pyrazine (Aldrich), 2,6-dichloro-pyrazine (Aldrich), 3-chloro-2,5-dimethylpyrazine (Aldrich), 2,4-dichloro-pyrimidine (Aldrich), 4,6-dichloro-pyrimidine (Aldrich), 4-chloro-2-methyl-pyrimidine (Chem. Ber. 1904, 37, 3641), 4-chloro-6-methyl-pyrimidine (Chem. Ber. 1899, 32, 2931), 4-chloro-2,6-dimethyl-pyrimidine (J. Am. Chem. Soc. 1946, 68, 1299), 4-chloro-2,6-bis(trifluoromethyl)-pyrimidine (J. Org. Chem. 1961, 26, 4504), 4-chloro-2-methylsulfanyl-pyrimidine (Aldrich), 4-chloro-2-methoxymethyl-pyrimidine (U.S. Pat. No. 5,215,990), 1-chloro-isoquinoline (J. Am. Chem. Soc. 1946, 68, 1299), 2-chloro-quinoline (Aldrich), 4-chloro-quinazoline (J. Am. Chem. Soc. 1909, 31, 509), 2-chloro-quinoxaline (U.S. Pat. No. 2,537,870), 2-chloro-3-methyl-quinoxaline (Aldrich), 2,6,7-trichloro-quinoxaline (J. Chem. Soc., Chem. Commun. 1956, 4731), 4-chloro-pteridine (J. Chem. Soc., Chem. Commun. 1954, 3832), 7-chloro-pteridine (J. Chem. Soc., Chem. Commun. 1954, 3832), and 6-chloro-9H-purine (Aldrich). Other compounds of formula 5-1 can be prepared using methods well known to those skilled in the art or by using methods analogous to those described in the foregoing references.

Compounds of formula 5-3 are prepared by the displacement reaction of a compound of formula 5-1 with an amine of the formula 5-2. The reaction is conducted in the presence of a non-aqueous base, prefeably an organic amine such as pyridine, 4-dimethylaminopyridine, triethylamine or N,N′-diisopropylethylamine; an inorganic base such as potassium or sodium carbonate or bicarbonate; or an alkaline metal alkoxide such as potassium t-butoxide. Alternatively, an excess of the reacting amine 5-2 can be used in lieu of the added base. In cases where the leaving group LV³ is unactivated, or in specific cases which will be recognized by those skilled in the art, the use of a transition-metal catalyst such as palladium(0), palladium (II), nickel(0) or nickel(II), along with phosphine-based ligands, such as 2,2′-bis(diphenylphosphino)-1,1′-binaphthyl (BINAP), may be required to effect reaction. More specific details concerning this reaction are available in the following references: J. Org. Chem. 1997, 62, 1264; J. Org. Chem. 1997, 62, 1568; SynLett 1997, 329. The reaction can be conducted in the absence of solvent or in a reaction inert solvent. Preferable reaction inert solvents include aqueous media, (C₁-C₄)alcohol, (C₂-C₆)glycol, a halocarbon, an aliphatic or aromatic hydrocarbon, an ether, a polar aprotic solvent, a ketone, or combinations thereof. The reaction time ranges from 15 minutes to 3 days and the reaction temperature ranges from 0° C. to 180° C. or to the reflux temperature of the solvent being used. The reactions are preferably conducted at ambient pressure.

In certain cases which will be recognized by those skilled in the art, transformations of existing functionality in Ar¹ of compound 5-3 may be necessary to produce compounds of formula 5-4. This pertains in particular to those cases where, for example, R⁹ in 5-3 contains an aromatic or heteroaromatic halide, (C₁-C₄)alkylsulfonate or triflate. Said compounds of formula 5-3 wherein Ar¹ contains up to two substituents selected from halide, (C₁-C₄)alkylsulfonate or triflate, may be converted to a compound of formula Ar¹ where said halide, (C₁-C₄)alkylsulfonate or triflate is transformed into another functional group by a reduction reaction or by a displacement reaction of said halide, (C₁-C₄)alkylsulfonate or triflate with a nucleophile. The reduction reaction is conducted with a reducing agent, preferably ammonium formate or hydrogen gas, in a reaction inert solvent. The reduction is conducted in the presence of a palladium catalyst at ambient pressure or under a hydrogen pressure of up to 50 psi. Preferred solvents include (C₁-C₄)alcohols such as methanol and ethanol, and ether solvents such as diethyl ether, dioxane and tetrahydrofuran. The nucleophilic displacement reaction may be conducted by adding the nucleophile directly or by pre-forming the nucleophile separately or in situ from a nucleophile precursor. Preferred nucleophiles include organoaluminum, organoboron, organocopper, organotin, organozinc or Grignard reagent; R¹¹-oxide or R¹¹-thioxide; or anilino where anilino is within the scope of R¹¹. It will be recognized by those skilled in the art that transition-metal catalysts may be required to effect reaction in certain displacement reactions. When required, such transition metal catalysts may include palladium(0), palladium(II), nickel(0), and nickel(II) complexes. Palladium(II) bis(diphenylphosphinobutane) dichloride is a preferred such catalyst. Additionally, an aqueous or non-aqueous base may be required in the displacement reaction. Preferred such bases include sodium carbonate, sodium hydride, triethylamine and sodium tert-butoxide. The reaction is conducted at ambient pressure in a reaction inert solvent such as a halocarbon, an aromatic or aliphatic hydrocarbon, an ether or a polar aprotic solvent or a combination thereof. In certain cases, a (C₁-C₄)alcohol is used as a solvent or co-solvent. The reaction temperature ranges from −20° C. to the reflux temperature of the solvent employed. The reaction time ranges from 1 hour to 3 days.

Optional protecting groups which may be present in compounds of formula 5-3 are removed according to methods set forth above, or according to methods well known to those skilled in the art, particularly as set forth in: Greene, T. W.; Wuts, P. G. M. Protective Groups in Organic Synthesis, 2^{nd} ed.; John Wiley and Sons Inc.: New York, 1991.

Compounds of formula 5-4 are prepared from the displacement reaction of amine 5-3 as described in Scheme 1, where the amine 5-3 is equivalent to R³—H. A representative set of amines of formula 5-3 which are commercially available or which can be prepared by a literature procedure include 1-phenyl-piperazine (Aldrich), 1-pyridin-2-yl-piperazine (Aldrich), 3-piperazin-1-yl-benzo[d]isoxazole (J. Med. Chem. 1986, 29, 359), 3-piperazin-1-yl-benzo[d]isothiazole (J. Med. Chem. 1986, 29, 359), 2-piperazin-1-yl-quinoxaline (J. Med. Chem. 1981, 24, 93), 1-naphthalen-2-yl-piperazine (cf. Tetrahedron Lett. 1994, 35, 7331), and 1-(3,5-dimethylphenyl)-piperazine (cf. Tetrahedron Lett. 1994, 35, 7331). Other compounds of formula 5-3 can be prepared using methods well known to those skilled in the art or by using methods analogous to those described in the foregoing references.

Alternatively, compounds of formula 5-4 can be prepared from reaction with compounds of formula 5-1 with compounds of formula 5-5 using conditions set forth above to prepare 5-3. Compounds of formula 5-5 can be prepared in a manner analogous to the method used to prepare compounds of formula 1-3.

Compounds of formula 5-4 wherein X is oxycarbonyl, vinylenylcarbonyl, oxy(C₁-C₄)alkylenylcarbonyl, (C₁-C₄)alkylenylcarbonyl, (C₃-C₄)alkenylcarbonyl, thio(C₁-C₄)alkenylcarbonyl, vinylenylsulfonyl or carbonyl(C₀-C₄)alkylenylcarbonyl; wherein said oxy(C₁-C₄)alkylenylcarbonyl, (C₁-C₄)alkylenylcarbonyl, (C₃-C₄)alkenylcarbonyl, and thio(C₃-C₄)alkenylcarbonyl in the definition of X are each optionally and independently substituted with up to two (C₁-C₄)alkyl, benzyl, or Ar; said vinylenylsulfonyl and said vinylenylcarbonyl in the definition of X are each optionally and independently substituted with up to three (C₁-C₄)alkyl, benzyl, or Ar are also prepared according to Scheme 5 above and particularly as described below.

Compounds of formula 5-4 where X is as defined in the immediately preceding paragraph are prepared by reacting a compound of formula 5-5 with a compound of formula 5-1 where R⁹ is described above, X is as defined in the immediately preceding paragraph and Lv³ is chloro. The reaction is conducted under anhydrous conditions in the presence of a non-aqueous base, which includes organic amines such as triethylamine, N,N′-diisopropylethylamine and pyridine and derivatives thereof. The reaction is generally conducted in a reaction inert solvent. Preferred solvents include halocarbon, aliphatic or aromatic hydrocarbon, ethers, ethyl acetate, pyridine and combinations thereof. The reaction time ranges from 15 minutes to 24 hours and the reaction temperature ranges from 0° C. to 80° C. or to the reflux temperature of the solvent being used. The reactions are preferably conducted at from 0° C. to ambient temperature and at ambient pressure. Removal of optional protecting groups is carried out as described in Scheme I.

Compounds of formula 5-4 wherein X is vinylenylcarbonyl, oxy(C₁-C₄)alkylenylcarbonyl, (C₁-C₄)alkylenylcarbonyl, (C₃-C₄)alkenylcarbonyl, thio(C₂-C₄)alkenylcarbonyl, or carbonyl(C₀-C₄)alkylenylcarbonyl,; wherein said oxy(C₁-C₄)alkylenylcarbonyl, (C₁-C₄)alkylenylcarbonyl, (C₃-C₄)alkenylcarbonyl, and thio(C₂-C₄)alkenylcarbonyl in the definition of X are each optionally and independently substituted with up to two (C₁-C₄)alkyl, benzyl, or Ar; and said vinylenylcarbonyl in the definition of X are each optionally and independently substituted with up to three (C₁-C₄)alkyl, benzyl, or Ar are also prepared according to Scheme 5 avove and particularly as described below.

Compounds of formula 5-4 are prepared by reacting a compound of formula 5-5 with a compound of formula R⁹—X—Lv³ where R⁹ is described above, X is as defined in the immediately preceding paragraph and Lv³ is OH. The reaction is conducted in the presence of coupling agents, preferably dicyclohexylcarbodiimide or 1-(3-dimethylaminopropyl)-3-ethylcarbodiimide hydrochloride as described in J. Amer. Chem. Soc. 1996, 118, 4952. The reaction is conducted in a reaction inert solvent. Preferred solvents include halocarbon, aliphatic or aromatic hydrocarbon and ethers. Especially preferred solvents include dichloromethane and chloroform. Other coupling agents that can be used are well known to those skilled in the art and include, but are not limited to, various phosphine reagents, ethyl chloroformate, and N-hydroxysuccinimide. These reagents and procedures are described in “Compendium of Organic Synthetic Methods” (Ed., I. T. Harrison and S. Harrison, John Wiley & Sons). Specific references include the following: J. Org. Chem, 1971, 36, 1305; Bull Soc. Chim. Fr., 1971, 3034; Bull. Chem. Soc. Japan, 1971, 44, 1373; Tetrahedron Lett., 1973, 28, 1595; Tetrahedron Lett., 1971, 26, 2967, and J. Med. Chem., 1968, 11, 534. Removal of optional protecting groups is carried out as described in Scheme I.

Compounds of formula 5-4 wherein X is a covalent bond and R⁹ is (C₃-C₇)cycloalkyl or Ar¹—(C₁-C₃)alkylenyl are also prepared according to Scheme 5 above and particularly as described below.

Compounds of formula 5-4 wherein X is a covalent bond and R⁹ is (C₃-C₇)cycloalkyl or Ar¹—(C₁-C₃)alkylenyl are prepared by reacting a compound of formula 5-1 wherein X is a covalent bond, R⁹ is (C₃-C₇)cycloalkyl or Ar¹—(C₁-C₃)alkylenyl and Lv³ is halo, methanesulfonate, p-toluenesulfonate or trifluoromethanesulfonate. The reaction is conducted under anhydrous conditions in the presence of a non-aqueous base, which includes organic amines such as triethylamine, N,N′-diisopropylethylamine and pyridine and derivatives thereof. The reaction is conducted in a reaction inert solvent. Preferred solvents for the reaction include halocarbons, aliphatic or aromatic hydrocarbons, ethers, ethyl acetate, pyridine and combinations thereof. The reaction time ranges from 15 minutes to 24 hours and the reaction temperature ranges from −20° C. to 80° C. or to the reflux temperature of the solvent being used. The reactions are preferably conducted at ambient temperature of the solvent being used and at ambient pressure. Removal of optional protecting groups is conducted as set forth in Scheme I.

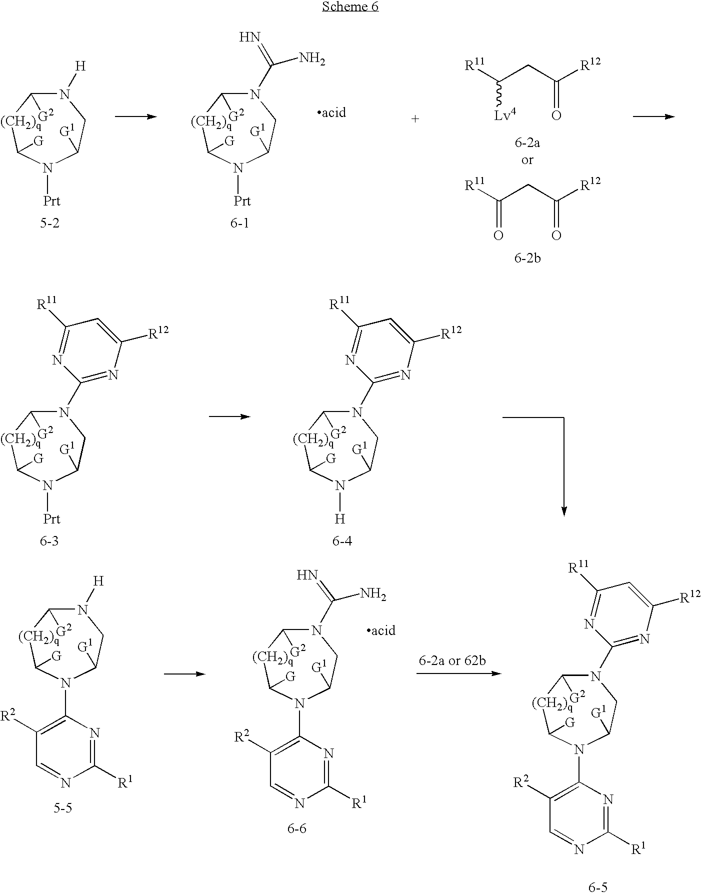

Compounds of formula 6-5 wherein G, G¹, G², q, R¹, R², R⁶, R⁷ and R⁸ are as defined above are prepared as set forth in Scheme 6 above and particularly as described below.

Compounds of formula 6-1 are prepared from an amine of the formula 5-2 where Prt is an optional amine protecting group selected from benzyl and CO₂R⁹⁰, where R⁹⁰ is selected from (C₁-C₄)alkyl, (C₁-C₄)allyl, trichloroethyl and benzyl substituted with up to two (C₁-C₄)alkoxy. The preferred procedure for preparing compounds of formula 6-1 can be found in Tetrahedron Lett. 1993, 48, 7767 or J. Org. Chem 1997, 62, 1540.

Compounds of formula 6-3 are prepared by condensation of β-diketones or , β-ketoesters of the formula 6-2b, where R¹¹ and R¹² are independently substituted as set forth above, or compounds of the formula 6-2a where Lv⁴ is, for example, hydroxy, chloro or dimethylamino with guanidines of the formula 6-1. The reaction is conducted in the presence of an aqueous or non-aqueous base, preferably potassium or sodium hydroxide, potassium or sodium (C₁-C₄)-alkoxide, triethylamine, pyridine, 4-dimethylaminopyridine, potassium or sodium carbonate or potassium or sodium bicarbonate. The reaction is conducted in a reaction inert solvent, preferably aqueous media, a (C₁-C₄)alcohol, a (C₂-C₆)dialcohol, an aromatic hydrocarbon, a polar aprotic solvent, or combinations thereof. The reaction time ranges from 2 hours to 3 days and the reaction temperature ranges from room temperature to reflux of the solvent employed. The reaction is preferably run at ambient pressure, but may be conducted at pressures up to 250 psi.

Removal of of optional protecting groups in compounds of formula 6-3 to afford compounds of formula 6-4 is accomplished as set forth above.

Compounds of formula 6-5 are prepared from the displacement reaction of amine 6-4 as described in Scheme 1, where the amine 6-4 is equivalent to R³—H.

Alternatively, compounds of formula 6-5 are prepared from compounds of formula 5-5 by formation of a compound of formula 6-6, and then by reaction with compounds of formula 6-2a or 6-2b under the conditions outlined above in Scheme 6. Removal of optional protecting groups is conducted as described in Scheme 1. Compounds of formula 5-5 are prepared as set forth above.

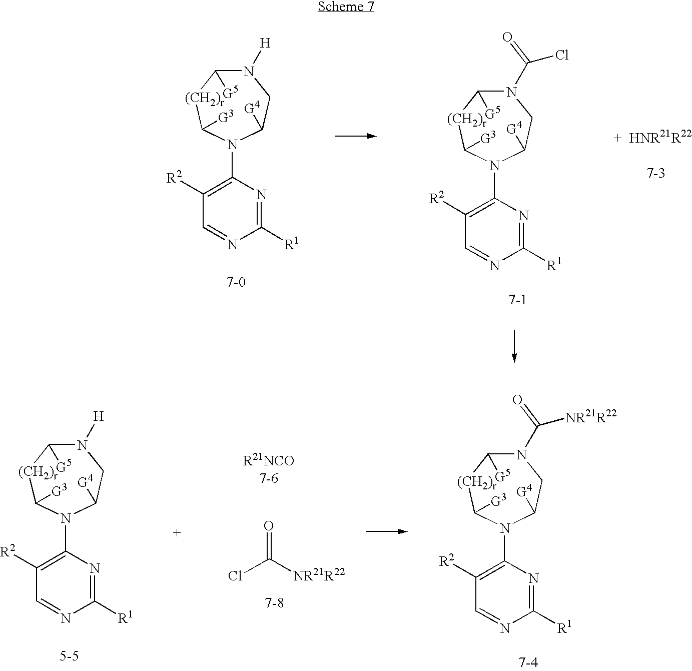

Compounds of formula 7-4 wherein G³, G⁴, G⁵, r, R¹, R², R¹⁸, R¹⁹ and R²⁰ are defined as set forth above are prepared as set forth in Scheme 7 and particularly as described below.

Compounds of formula 7-1 are prepared by reaction of an amine of the formula 7-0 with phosgene or a phosgene equivalent such as triphosgene. Compounds of 7-1 wherein the chloro group is replaced by an imidazolyl group are also useful in this reaction. Such compounds are prepared by reaction of an amine of formula 7-0 with carbonyl diimidazole. The reaction is conducted under anhydrous conditions in the presence of a nonaqueous base. Preferred such bases include triethylamine and other tertiary amines and pyridine and derivatives thereof. The reaction is conducted in a reaction inert solvent at −78° C. to 80° C. or at the reflux temperature of the solvent being used for 15 minutes to 24 hours. Preferred solvents for this reaction include a halocarbon, an aliphatic or aromatic hydrocarbon, an ether, ethyl acetate, pyridine and combinations thereof. The reactions are preferably conducted at from 0° C. to ambient temperature and at ambient pressure.

Compounds of formula 7-4 are prepared by reaction of carbamoyl chlorides of the formula 7-1 with amines of the formula 7-3, where R²¹ and R²² are defined above. The reaction can be conducted in the absence of solvent, or in a reaction inert solvent. Preferred such solvents include aqueous media, a (C₁-C₄)alcohol, a (C₂-C₆)dialcohol, an aromatic or aliphatic hydrocarbon, a halocarbon, an ether, a polar aprotic solvent, a ketone, pyridine or combinations thereof. The reaction time ranges from 15 minutes to 3 days and the reaction temperature ranges from 0° C. to the reflux temperature of the solvent being used. The reaction is preferably conducted at ambient pressure. It will be recognized by those skilled in the art that addition of a base may be required to effect reaction. In those cases, preferred bases include potassium or sodium hydroxide, triethylamine and other tertiary amines, pyridine and its derivatives and inorganic bases such as sodium or potassium carbonate and sodium or potassium bicarbonate. Removal of optional hydroxyl protecting groups contained in R¹ is carried out according to methods set forth in Scheme 1.

Alternatively, compounds of formula 7-4 are prepared from compounds of formula 7-0 by reaction with isocyanates of the formula 7-6 or with carbamoyl chlorides of the formula 7-8. Said isocyanates are commercially available, known in the literature, or synthesized under standard conditions known to those skilled in the art, particularly as described in March, J. Advanced Organic Chemistry, 3^{rd} ed.; John Wiley and Sons Inc.: New York, 1985, p 1166. A preferred method of forming such isocyanates is the Curtius rearrangement of a suitable acyl azide. Said carbamoyl chlorides are synthesized using methods analogous to that described for the preparation of compounds of formula 7-1 in Scheme 7.Removal of optional hydroxyl protecting groups contained in R¹ is carried out according to methods set forth in Scheme 1.

Compounds of formula I containing the radical R^{3c} are prepared according to the procedures set forth in Scheme 7 using the corresponding starting materials and reagents.

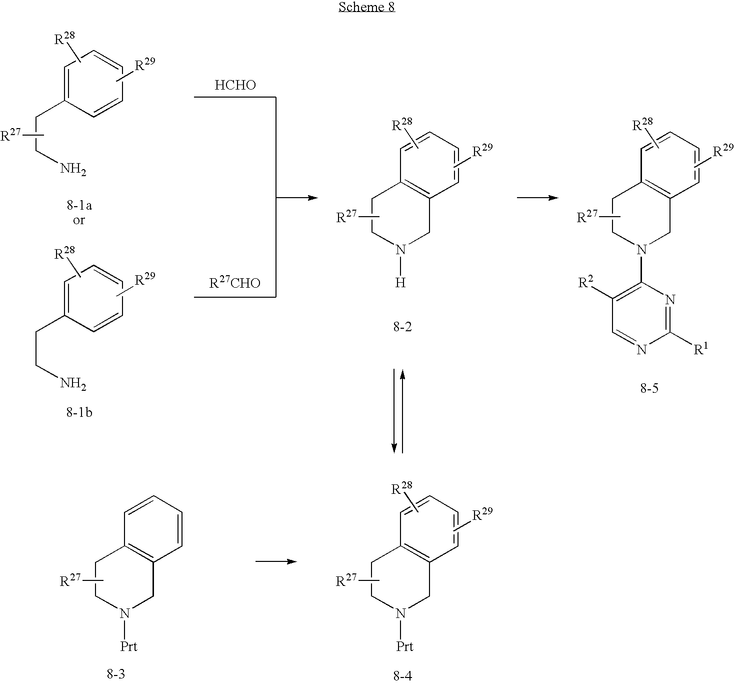

Compounds of formula 8-5 are prepared as set forth in Scheme 8 and particularly as described below.

Compounds of formula 8-2 are readily prepared from commercially available phenethylamines of formula 8-1a and formaldehyde or an aldehyde of the formula R²⁷—CHO under Pictet-Spengler conditions. The Pictet-Spengler reaction is reviewed in Chem. Rev. 1995, 95, 1797. A similar route route to 1,2,3,4-tetrahydroisoquinolines using the Bischler-Napieralski reaction, as disclosed in March, J. Advanced Organic Chemistry, 3^{rd} ed.; John Wiley and Sons.: New York, 1985, 495, followed by standard reduction of the imine formed may also be employed.

Compounds of formula 8-4 are prepared from compounds of formula 8-3 by aromatic electrophilic substitution using the appropriate electrophile. A general reference for this type of reaction can be found in March, J. Advanced Organic Chemistry, 3^{rd} ed.; John Wiley and Sons.: New York, 1985, 447-511.

Compounds of formula 8-2 are also prepared by removal of the protecting group from a compound of formula 8-4. Preferably the protecting group is trifluoroacetamide which may be removed under basic conditions using inorganic hydroxides or carbonates in a reaction inert solvent. Suitable such solvents include (C₁-C₄)alcohols and preferably methanol. Optionally, one or more co-solvents, preferably selected from water, tetrahydrofuran and dioxane may be employed. The reaction time ranges from 15 minutes to 24 hours and the reaction temperature ranges from 0° C. to 100° C. or to the reflux temperature of the solvent or solvent system being used. The reaction is preferably conducted at ambient temperature. Other conditions for deprotection of trifluoroacetamides and deprotection conditions for other suitable protecting groups can be found in Greene, T. W.; Wuts, P. G. M. Protective Groups in Organic Synthesis, 2^{nd} ed.; John Wiley and Sons Inc.: New York, 1991.

Compounds of formula 8-4 are prepared by adding a protecting group to compounds of formula 8-2. Preferably the protecting group is trifluoroacetamide or tert-butoxycarbonyl (BOC). The protecting group is attached by reaction of a compound of formula 8-2 with trifluoroacetyl chloride or di-tert-butyl dicarbonate or an equivalent thereof in the presence of a base, preferably triethylamine or pyridine. The reaction is conducted in a reaction inert solvent. Preferred such solvents include ethers such as tetrahydrofuran, diethyl ether, dioxane or dimethoxyethane; a halocarbon such as dichloromethane, chloroform or carbon tetrachloride; and aromatic or aliphatic hydrocarbons such as benzene, toluene or hexanes. The reaction time ranges from 15 minutes to 3 days and the reaction temperature ranges from 0° C. to the reflux temperature of the solvent being used. The reaction is preferably conducted at ambient pressure. Other conditions for protection of amines with trifluoroacetamides or tert-butoxycarbonyl groups as well as other suitable protecting groups can be found in Greene, T. W.; Wuts, P. G. M. Protective Groups in Organic Synthesis, 2^{nd} ed.; John Wiley and Sons Inc.: New York, 1991.

Manipulation of the substituents R²⁸ and R²⁹ is carried out to provide isoquinolines with altered substitution. Preferably, transition metal-catalyzed cross-coupling of a compound of formula 8-4 where R²⁸ or R²⁹ is bromide or triflate is employed to afford compounds of formula 8-4 wherein R²⁸ or R²⁹ are as set forth above. This reaction is conducted according to methods well known to those skilled in the art, particularly as set forth in Tetrahedron, 1998, 54, 263 for Stille and Suzuki Reactions and in Acc. Chem. Res. 1998, 31, 805 for Buchwald Amination Reactions.

Compounds of formula 8-5 are prepared from the displacement reaction of amine 8-2 as described in Scheme 1, where the amine 8-2 is equivalent to R³—H.

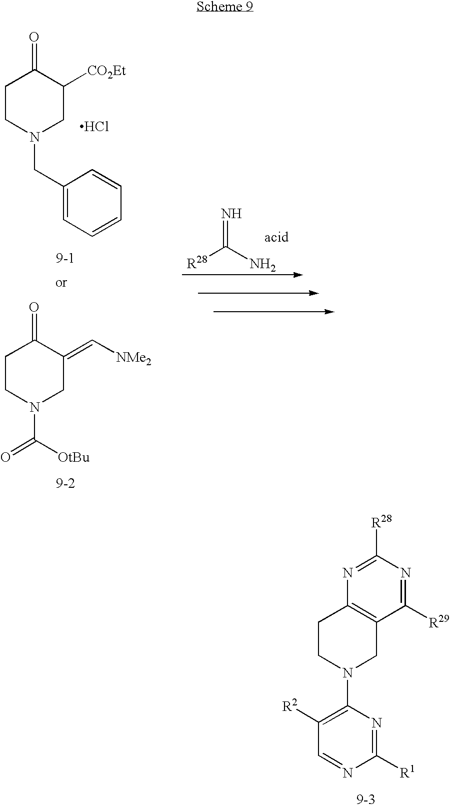

Compounds of formula 9-3 are prepared according to the general procedures set forth in Scheme 2 starting from ethyl 1-benzyl-4-oxo-3-piperidine carboxylate hydrochloride (9-1). In certain cases, where R²⁹ is H, N-tertbutoxycarbonyl-3-(dimethylaminomethylene)-4-piperidone (9-2, Chemical Abstracts 121:157661) is used as the starting material.

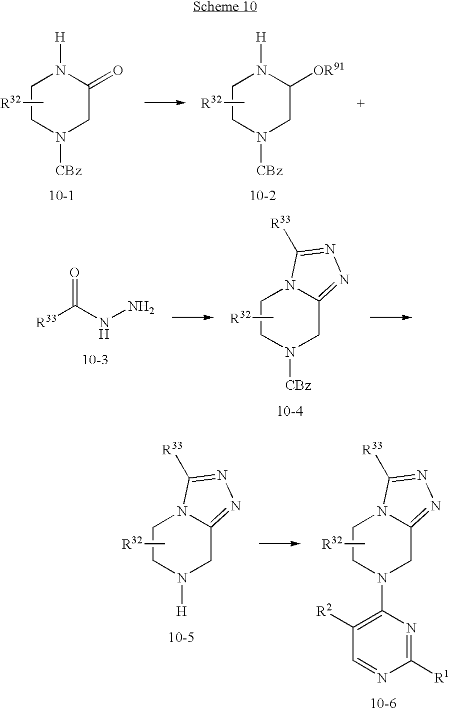

Compounds of formula 10-6 wherein R¹, R², R³² and R³³ are as defined above are prepared as set forth in Scheme 10 and more particularly as described below.

Compounds of formula 10-2 where R⁹¹ is (C₁-C₄)alkyl are prepared by reacting a compound of formula 10-1, where Cbz is benzyloxycarbonyl, with an O-alkylating agent. A preferred compound of formula 10-1 is 3-oxo-piperazine-1-carboxylic acid benzyl ester. A preferred O-alkylating agent is triethyloxonium tetrafluoroborate. The reaction is conducted at ambient pressure in a reaction inert solvent. Preferred solvents include an aromatic or aliphatic hydrocarbons, halocarbons and ethers. Dichloromethane is especially preferred. The reaction time ranges from 2 hours to 3 days and the reaction temperature ranges from −100° C. to ambient temperature.

Compounds of formula 10-4 are prepared by condensation of a compound of formula 10-2 with a compound of formula 10-3. Said compounds of formula 10-3 are commercially available, are known in the literature, or are readily prepared via standard amidation of hydrazine and an activated carboxylic acid, such as a carboxylic acid chloride. Such reactions are well known by those skilled in the art. The condensation reaction is preferably run at ambient pressure, although higher pressures up to 250 psi may be employed if necessary. The reaction is conducted in a reaction inert solvent, preferably selected from (C₁-C₄)alcohols, aromatic or aliphatic hydrocarbons, polar aprotic media, halocarbons and ethers, or combinations thereof. The reaction is conducted at temperatures ranging from ambient temperature to 180° C. The reaction times are from 2 hours to 3 days.

Compounds of formula 10-5 are prepared form compounds of formula 10-4 via Lewis acid-catalyzed cleavage or hydrogenolysis of the Cbz carbamate under standard conditions which are well known to those skilled in the art, particularly as set forth in Greene, T. W.; Wuts, P. G. M. Protective Groups in Organic Synthesis, 2^{nd} ed.; John Wiley and Sons Inc.: New York, 1991, pp 335-338.

Compounds of formula 10-6 are prepared from the displacement reaction of an amine of the formula 10-5 as described in Scheme 1, where the amine 10-5 is equivalent to R³—H.

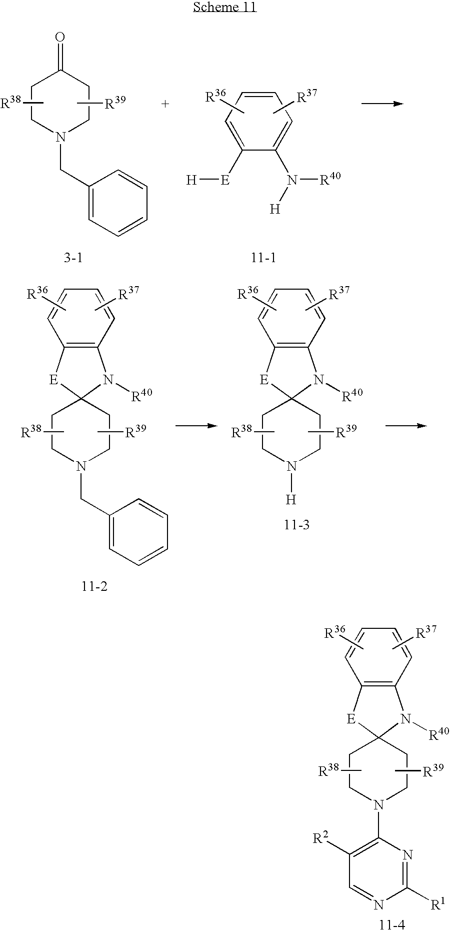

Compounds of formula 11-4 wherein R¹, R², R³⁶, R³⁷, R³⁸, R³⁹ and R⁴⁰ are as defined above are prepared as set forth in Scheme 11 and more particularly as described below.

Where R³⁸ and R³⁹ are hydrogen, 1-benzyl-4-piperidone (3-1), available from Aldrich, is condensed with a compound of formula 11-1, which are either commercially available or well known to those skilled in the art, to give compounds of formula 11-2. Where R³⁸ and R³⁹ are not hydrogen, compounds of formula 3-1 can be prepared according to methods well known to those skilled in the art. The reaction is conducted at ambient pressure in the absence of solvent or in a reaction inert solvent. Preferred solvents include (C₁-C₄)alcohols, aromatic or aliphatic hydrocarbons, polar aprotic solvents, halocarbons and ethers. The reaction time ranges from 2 hours to 3 days and the reaction temperature ranges from ambient temperature to the reflux temperature of the solvent being employed. More specific conditions can be found in Indian J. Chem. 1976, 14B, 984 and J. Chem. Soc., Perkin Trans. 1 1984, 2465.

Compounds of formula 11-3 are prepared by removal of the benzyl protecting group from a compound of formula 11-2 in a manner analogous to the method employed for the preparation of compounds of 2-6 described above.

Compounds of formula 11-4 are prepared by the displacement reaction of an amine of the formula 11-3 as described in Scheme 1, where the amine 11-3 is equivalent to R³—H.

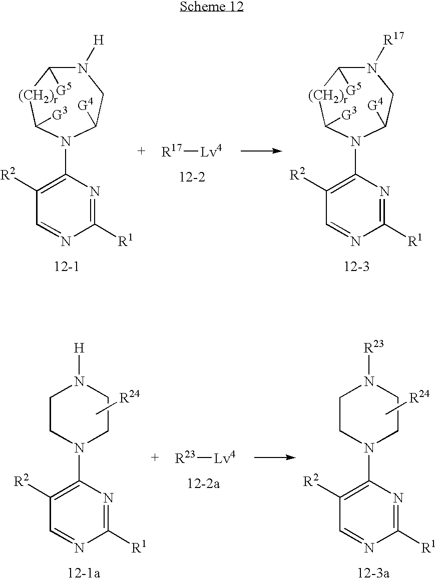

Compounds of formula 12-3 and 12-3a where R¹⁷ and R²³ are (C₁-C₆)alkoxycarbonyl, (C₁-C₆)alkylcarbonyl, Ar²-carbonyl, (C₁-C₆)alkylsulfonyl, Ar²-sulfonyl, or Ar²-sulfinyl are prepared according to Scheme 12 above and particularly as set forth below.

Compounds of formula 12-3 and 12-3a where R¹⁷ and R²³ are as defined in the immediately preceding paragraph are prepared by condensation with a compound of formula 12-2 and 12-2a, wherein Lv⁴ is chloro, respectively. Examples of compounds of formula 12-2 and 12-2a include (C₁-C₆)alkoxyCOCl, (C₁-C₆)alkylCOCl, Ar²—COCl, (C₁-C₆)alkylSO₂Cl, Ar²—SO₂Cl, or Ar²—SOCl. The reaction is conducted under anhydrous conditions in the presence of a non-aqueous base, which includes organic amines such as triethylamine, N,N′-diisopropylethylamine and pyridine and derivatives thereof. The reaction is conducted in a reaction inert solvent. Preferred solvents for the reaction include halocarbon, aliphatic or aromatic hydrocarbon, ethers, ethyl acetate, pyridine and combinations thereof. The reaction time ranges from 15 minutes to 24 hours and the reaction temperature ranges from 0° C. to 80° C. or to the reflux temperature of the solvent being used. The reactions are preferably conducted at from 0° C. to ambient temperature and at ambient pressure. Removal of optional protecting groups is carried out as described in Scheme I.

Compounds of formula 12-3 and 12-3a wherein R¹⁷ and R²³ are (C₁-C₆)alkylcarbonyl or Ar²-carbonyl are also prepared according to Scheme 12 above and particularly as described below.

Compounds of formula 12-3 and 12-3a wherein R¹⁷ and R²³ are (C₁-C₆)alkylcarbonyl or Ar²-carbonyl are prepared by a condensation reaction with a compound of formula 12-2 or 12-2a, respectively, wherein Lv⁴ is hydroxy in the presence of coupling agents such as dicyclohexylcarbodiimide or 1-(3-dimethylaminopropyl)-3-ethylcarbodiimide hydrochloride. The reaction is conducted in a reaction inert solvent. Preferred solvents include halocarbon, aliphatic/aromatic hydrocarbons and ethers. Especially preferred solvents include dichloromethane and chloroform. Other coupling agents that can be used are well known to those skilled in the art and include, but are not limited to, various phosphine reagents, ethyl chloroformate, and N-hydroxysuccinimide. Removal of optional protecting groups is carried out as described in Scheme I.

Compounds of formula 12-3 where R¹⁷ is (C₁-C₆)alkyl are also prepared according to Scheme 12 and particularly as described below.

Compounds of formula 12-3 where R¹⁷ is (C₁-C₆)alkyl are prepared by reacting a compound of formula 12-1 with a compound of formula 12-2 where R¹⁷ is (C₁-C₄)alkyl and Lv⁴ is Cl, Br, I, methanesulfonyloxy, p-toluenesulfonyloxy or trifluoromethanesulfonyloxy. The reaction is conducted under anhydrous conditions in the presence of a nonaqueous base, which includes organic amines such as triethylamine, Hunig's base and pyridine and derivatives thereof. The reaction is conducted in a reaction inert solvent. Preferred solvents for the reaction include halocarbons, aliphatic or aromatic hydrocarbons, ethers, ethyl acetate, pyridine and combinations thereof. The reaction time ranges from 15 minutes to 24 hours and the reaction temperature ranges from ambient temperature to 80° C. or to the reflux temperature of the solvent being used. The reactions are preferably conducted at ambient temperature and pressure.

The starting materials and reagents for the above described compounds are also readily available or can be easily synthesized by those skilled in the art using conventional methods of organic synthesis. For example, many of the compounds used herein are related to, or are derived from, compounds found in nature, in which there is a large scientific interest and commercial need, and accordingly many such compounds are commercially available or are reported in the literature or are easily prepared from other commonly available substances by methods which are reported in the literature.

The sorbitol dehydrogenase inhibiting activity of the sorbitol dehydrogenase inhibitors used in the combinations, pharmaceutical compositions, methods and kits of this invention may be determined according to procedures well known to those skilled in the art and particularly as set forth in commonly assigned U.S. patent application Ser. No. 09/538,039. Methods for determining the HMG-CoA inhibiting activity of the statins used in the combinations, pharmaceutical compositions, methods and kits of this invention are well known and may be achieved by following the procedures disclosed, for example, in U.S. Pat. No. 4,681,893.

The above assays demonstrating the effectiveness of sorbitol dehydrogenase inhibitors, prodrugs thereof and pharmaceutically acceptable acid addition salts of said sorbitol dehydrogenase inhibitors and said prodrugs, and statins, prodrugs thereof and pharmaceutically acceptable salts of said statins and said prodrugs in the treatment of atherosclerosis and diabetic complications also provide a means whereby the activities of the compounds of this invention can be compared between themselves and with the activities of other known compounds. The results of these comparisons are useful for determining dosage levels in mammals, including humans, for the treatment of such diseases.

The effect of a pharmaceutical composition comprising a statin and a sorbitol dehydrogenase inhibitor of the present invention may be examined by using one or more of the published models of diabetic complications well known in the art. The pharmaceutical compositions of the present invention are particularly useful for the prevention of, reducing the development of, or reversal of, deficits in nerve function found in diabetic patients, and therefore are particularly useful in the treatment of diabetic neuropathy. This may be demonstrated, for example, by measuring markers such as nerve conduction velocity, nerve amplitude, quantitative sensory testing, autonomic function testing and morphometric changes. Experimentally, studies analogous to those described in Diabetologia, 1992, Vol. 35, pages 12-18 and 1994, Vol. 37, pages 651-663 may be carried out. Further, studies analogous to those described in International Patent Application Publication No. WO99/02189.

In general, the sorbitol dehydrogenase inhibitors used in the combinations, pharmaceutical compositions, methods and kits of this invention, and particularly the compounds of Formula I, and their pharmaceutically acceptable salts, will be administered at dosages between about 0.001 and about 100 mg/kg body weight of the subject to be treated per day, preferably from about 0.01 mg/kg to about 10 mg/kg, in single or divided doses. However, some variation in dosage will necessarily occur depending upon the condition of the subject being treated. The person responsible for administration will, in any event, determine the appropriate dose for the individual subject.

The following dosage amounts and other dosage amounts set forth elsewhere in this description and in the appendant claims are for an average human subject having a weight of about 65 kg to about 70 g. The skilled practitioner will readily be able to determine the dosage amount required for a subject whose weight falls outside the 65 kg to 70 kg range, based upon the medical history of the subject. All doses set forth herein, and in the appendant claims, are daily doses.

In general, in accordance with this invention, the above statins are administered in the following dosage amounts:

atorvastatin, generally about 2.5 mg to about 160 mg and, preferably, atorvastatin is administered in a dosage of about 10 mg to about 80 mg;

simvastatin, generally about 2.5 mg to about 160 mg and preferably about 10 mg to about 40 mg;

pravastatin, generally about 2.5 mg to about 160 mg and preferably about 10 mg to about 40 mg;

cerivastatin, generally about 25 μg to about 5 mg and preferably about 1 mg to about 3.2 mg;

fluvastatin, generally about 2.5 mg to about 160 mg and preferably about 20 mg to about 80 mg; and

lovastatin, generally about 2.5 mg to about 160 mg and preferably about 10 mg to about 80 mg.

It will be recognized by a skilled person that the free base form or other salt forms of the above statins may be used in this invention. In particular, the calcium salt of atorvastatin is an especially preferred statin for use in the combinations of this invention. Calculation of the dosage amount for these other forms of the free base form or other salt forms of atorvastatin calcium is easily accomplished by performing a simple ratio relative to the molecular weights of the species involved.

For oral administration a pharmaceutical composition can take the form of solutions, suspensions, tablets, pills, capsules, powders, and the like. Tablets containing various excipients such as sodium citrate, calcium carbonate and calcium phosphate are employed along with various disintegrants such as starch and preferably potato or tapioca starch and certain complex silicates, together with binding agents such as polyvinylpyrrolidone, sucrose, gelatin and acacia. Additionally, lubricating agents such as magnesium stearate, sodium lauryl sulfate and talc are often very useful for tabletting purposes. Solid compositions of a similar type are also employed as fillers in soft and hard-filled gelatin capsules; preferred materials in this connection also include lactose or milk sugar as well as high molecular weight polyethylene glycols. When aqueous suspensions and/or elixirs are desired for oral administration, the compounds of this invention can be combined with various sweetening agents, flavoring agents, coloring agents, emulsifying agents and/or suspending agents, as well as such diluents as water, ethanol, propylene glycol, glycerin and various like combinations thereof.

The combinations of this invention may also be adminstered in a controlled release formulation such as a slow release or a fast release formulation. Such controlled release formulations of the combination of this invention may be prepared using methods well known to those skilled in the art. The method of adminstration will be determined by the attendant physician or other person skilled in the art after an evaluation of the subject's condition and requirements. The generally preferred formulation of atorvastatin is Lipitor®.

The combinations of this invention may also be administered in parenteral form. For parenteral administration, solutions in sesame or peanut oil or in aqueous propylene glycol can be employed, as well as sterile aqueous solutions of the corresponding water-soluble salts. Such aqueous solutions may be suitably buffered, if necessary, and the liquid diluent first rendered isotonic with sufficient saline or glucose. These aqueous solutions are especially suitable for intravenous, intramuscular, subcutaneous and intraperitoneal injection purposes. In this connection, the sterile aqueous media employed are all readily obtainable by standard techniques well-known to those skilled in the art.

Methods of preparing various pharmaceutical compositions with a certain amount of active ingredient are known, or will be apparent in light of this disclosure, to those skilled in this art. For examples, see Remington's Pharmaceutical Sciences, Mack Publishing Company, Easton, Pa., 19th Edition (1995).

Pharmaceutical compositions according to the invention may contain 0.1%-95% of the compound(s) of this invention, preferably 1%-70%. In any event, the composition or formulation to be administered will contain a quantity of a compound(s) according to the invention in an amount effective to treat the condition or disease of the subject being treated.

The two different compounds of this combination of this invention can be co-administered simultaneously or sequentially in any order, or as a single pharmaceutical composition comprising a compound of Formula I and an aldose reductase inhibitor as described above or a glycogen phosphorylase inhibitor as described above or a cardiovascular agent.

Since the present invention has an aspect that relates to the treatment of the disease/conditions described herein with a combination of active ingredients which may be administered separately, the invention also relates to combining separate pharmaceutical compositions in kit form. The kit comprises two separate pharmaceutical compositions: a sorbitol dehydrogenase inhibitor, a prodrug thereof or a pharmaceutically acceptable salt of said sorbitol dehydrogenase inhibitor or prodrug and a statin, a prodrug thereof or a pharmaceutically acceptable salt of said statin or prodrug. The kit comprises means for containing the separate compositions such as a container, a divided bottle or a divided foil packet. Typically the kit comprises directions for the administration of the separate components. The kit form is particularly advantageous when the separate components are preferably administered in different dosage forms (e.g., oral and parenteral), are administered at different dosage intervals, or when titration of the individual components of the combination is desired by the prescribing physician.

An example of such a kit is a so-called blister pack. Blister packs are well known in the packaging industry and are being widely used for the packaging of pharmaceutical unit dosage forms (tablets, capsules, and the like). Blister packs generally consist of a sheet of relatively stiff material covered with a foil of a preferably transparent plastic material. During the packaging process recesses are formed in the plastic foil. The recesses have the size and shape of the tablets or capsules to be packed. Next, the tablets or capsules are placed in the recesses and the sheet of relatively stiff material is sealed against the plastic foil at the face of the foil which is opposite from the direction in which the recesses were formed. As a result, the tablets or capsules are sealed in the recesses between the plastic foil and the sheet. Preferably the strength of the sheet is such that the tablets or capsules can be removed from the blister pack by manually applying pressure on the recesses whereby an opening is formed in the sheet at the place of the recess. The tablet or capsule can then be removed via said opening.

It may be desirable to provide a memory aid on the kit, e.g., in the form of numbers next to the tablets or capsules whereby the numbers correspond with the days of the regimen which the tablets or capsules so specified should be ingested. Another example of such a memory aid is a calendar printed on the card, e.g., as follows “First Week, Monday, Tuesday, . . . etc . . . Second Week, Monday, Tuesday, . . . ” etc. Other variations of memory aids will be readily apparent. A “daily dose” can be a single tablet or capsule or several pills or capsules to be taken on a given day. Also, a daily dose of the sorbitol dehydrogenase inhibitor can consist of one tablet or capsule while a daily dose of the statin can consist of several tablets or capsules and vice versa. The memory aid should reflect this.

In another specific embodiment of the invention, a dispenser designed to dispense the daily doses one at a time in the order of their intended use is provided. Preferably, the dispenser is equipped with a memory-aid, so as to further facilitate compliance with the regimen. An example of such a memory-aid is a mechanical counter which indicates the number of daily doses that has been dispensed. Another example of such a memory-aid is a battery-powered micro-chip memory coupled with a liquid crystal readout, or audible reminder signal which, for example, reads out the date that the last daily dose has been taken and/or reminds one when the next dose is to be taken.

It should be understood that the invention is not limited to the particular embodiments described herein, but that various changes and modifications may be made without departing from the spirit and scope of this novel concept as defined by the following claims.

1. A pharmaceutical composition comprising:
a. an amount of a statin, a prodrug thereof or a pharmaceutically acceptable salt of said statin or said prodrug; and
b. an amount of a sorbitol dehydrogenase inhibitor, a prodrug thereof or a pharmaceutically acceptable salt of said sorbitol dehydrogenase inhibitor or said prodrug.

2. A pharmaceutical composition of claim 1 additionally comprising a pharmaceutically acceptable carrier or diluent.

3. A pharmaceutical composition of claim 2 wherein said statin is atorvastatin, simvastatin, pravastatin, rivastatin, mevastatin, fluindostatin, velostatin, fluvastatin, dalvastatin, dihydrocompactin, compactin, lovastatin, a prodrug thereof or a pharmaceutically acceptable salt of said statin or said prodrug.

4. A pharmaceutical composition of claim 3 wherein said statin is atorvastatin, rivastatin, simvastatin, pravastatin, mevastatin, lovastatin, a prodrug thereof or a pharmaceutically acceptable salt of said statin or said prodrug.

5. A pharmaceutical composition of claim 4 wherein said statin is atorvastatin or atorvastatin calcium.

6. A pharmaceutical composition of claim 2 wherein said sorbitol dehydrogenase inhibitor is a compound of Formula I, 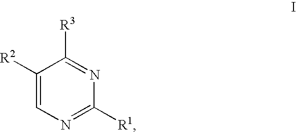
a prodrug thereof or a pharmaceutically acceptable salt of said compound or said prodrug, wherein:
R¹ is formyl, acetyl, propionyl, carbamoyl or —C(OH)R⁴R⁵;
R⁴ and R⁵ are each independently hydrogen, methyl, ethyl or hydroxy-(C₁-C₃)alkyl;
R² is hydrogen, (C₁-C₄)alkyl or (C₁-C₄)alkoxy;
R³ is a radical of the formula 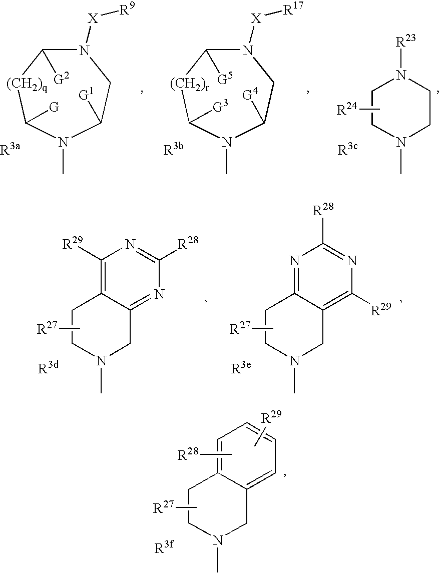
R¹², R¹³ and R¹⁴ is optionally substituted with up to two substituents independently selected from (C₁-C₄)alkyl; said pyrrolidinyl in the definition of R¹¹, R¹², R¹³ and R¹⁴ is optionally substituted with up to two substituents independently selected from hydroxy, hydroxy-(C₁-C₃)alkyl, (C₁-C₄)alkoxy-(C₁-C₄)alkyl, (C₁-C₄)alkyl optionally substituted with up to five fluoro and (C₁-C₄)alkoxy optionally substituted with up to five fluoro; said piperazinyl in the definition of R¹¹, R¹², R¹³ and R¹⁴ is optionally substituted with up to three substituents independently selected from (C₁-C₄)alkoxy-(C₁-C₄)alkyl, hydroxy-(C₁-C₃)alkyl, phenyl, pyridyl, (C₀-C₄)alkylsulfamoyl, (C₁-C₄)alkyl optionally substituted with up to five fluoro and (C₁-C₄)alkoxy optionally substituted with up to five fluoro; said triazolyl in the definition of R¹¹, R¹², R¹³ and R¹⁴ is optionally substituted with hydroxy, halo, hydroxy-(C₁-C₄)alkyl, (C₁-C₄)alkoxy-(C₁-C₄)alkyl, (C₁-C₄)alkyl optionally substituted with up to five fluoro and (C₁-C₄)alkoxy optionally substituted with up to five fluoro; said tetrazolyl in the definition of R¹¹, R¹², R¹³ and R¹⁴ is optionally substituted with hydroxy-(C₂-C₃)alkyl or (C₁-C₄)alkyl optionally substituted with up to five fluoro; and said phenyl and pyridyl which are optionally substituted on piperazine in the definition of R¹¹, R¹², R¹³ and R¹⁴ are optionally substituted with up to three hydroxy, halo, hydroxy-(C₁-C₄)alkyl, (C₁-C₄)alkoxy-(C₁-C₄)alkyl, (C₁-C₄)alkyl optionally substituted with up to five fluoro and (C₁-C₄)alkoxy optionally substituted with up to five fluoro; or
R¹¹ and R¹² are taken together on adjacent carbon atoms and are —CH₂OC(CH₃)₂OCH₂— or —O—(CH₂)ₚ—O—, and R¹³ and R¹⁴ are taken separately and are each independently hydrogen or (C₁-C₄)alkyl;
p is 1, 2 or 3;
R¹⁵ and R¹⁶ are taken separately and are each independently hydrogen, (C₁-C₄)alkyl optionally substituted with up to five fluoro; or R¹⁵ and R¹⁶ are taken separately and R¹⁵ is hydrogen and R¹⁶ is (C₃-C₆)cycloalkyl, hydroxy-(C₁-C₃)alkyl, phenyl, pyridyl, pyrimidyl, thienyl, furanyl, thiazolyl, oxazolyl, imidazolyl, benzothiazolyl or benzoxazolyl; or R¹⁵ and R¹⁶ are taken together and are (C₃-C₆)alkylene;
G³, G⁴ and G⁵ are taken separately and are each hydrogen; r is 0; R¹⁸ is hydrogen, (C₁-C₄)alkyl, (C₁-C₄)alkoxycarbonyl, (C₁-C₄)alkoxy-(C₁-C₄)alkyl, hydroxy-(C₁-C₄)alkyl or phenyl optionally independently substituted with up to three hydroxy, halo, hydroxy-(C₁-C₄)alkyl, (C₁-C₄)alkoxy-(C₁-C₄)alkyl, (C₁-C₄)alkyl or (C₁-C₄)alkoxy, wherein said (C₁-C₄)alkyl in the definition of R⁶ and said (C₁-C₄)alkoxy in the definition of R⁶ are optionally and independently substituted with up to five fluoro; and R¹⁹ and R²⁰ are each independently (C₁-C₄)alkyl; or
G³, G⁴ and G⁵ are taken separately and are each hydrogen; r is 1; R¹⁸ is hydrogen, (C₁-C₄)alkyl, (C₁-C₄)alkoxycarbonyl, (C₁-C₄)alkoxy-(C₁-C₄)alkyl, hydroxy-(C₁-C₄)alkyl or phenyl optionally independently substituted with up to three hydroxy, halo, hydroxy-(C₁-C₄)alkyl, (C₁-C₄)alkoxy-(C₁-C₄)alkyl, (C₁-C₄)alkyl or (C₁-C₄)alkoxy, wherein said (C₁-C₄)alkyl in the definition of R⁶ and said (C₁-C₄)alkoxy in the definition of R⁶ are optionally and independently substituted with up to five fluoro; and R¹⁹ and R²⁰ are each independently hydrogen or (C₁-C₄)alkyl; or
G³ and G⁴ are taken together and are (C₁-C₃)alkylene; r is 0 or 1; and R¹⁸, R¹⁹, R²⁰ and G⁵ are hydrogen; or
G⁴ and G⁵ are taken together and are (C₁-C₃)alkylene; r is 0 or 1; and R¹⁸, R¹⁹, R²⁰ and G³ are hydrogen;
R¹⁷ is SO₂NR²¹R²², CONR²¹R²², (C₁-C₆)alkoxycarbonyl, (C₁-C₆)alkylcarbonyl, Ar²-carbonyl, (C₁-C₆)alkylsulfonyl, (C₁-C₆)alkylsulfinyl, Ar²-sulfonyl, Ar²-sufonyl and (C₁-C₆)alkyl;
R²¹ and R²² are taken separately and are each independently selected from hydrogen, (C₁-C₆)alkyl, (C₃-C₇)cycloalkyl and Ar²—(C₀-C₄)alkylenyl; or
R²¹ and R²² are taken together with the nitrogen atom to which they are attached to form azetidinyl, pyrrolidinyl, piperidinyl, piperazinyl, morpholinyl, azepinyl, azabicyclo[3.2.2]nonanyl, azabicyclo[2.2.1]heptyl, 6,7-dihydro-5H-dibenzo[c,e]azepinyl, 1,2,3,4-tetrahydro-isoquinolyl or 5,6,7,8-tetrahydropyrido[4,3-d]pyrimidyl; said azetidinyl in the definition of R²¹ and R²² is optionally substituted independently with one substituent selected from hydroxy, amino, hydroxy-(C₁-C₄)alkyl, (C₁-C₄)alkoxy-(C₁-C₄)alkyl, (C₁-C₄)alkyl optionally substituted with up to five fluoro and (C₁-C₄)alkoxy optionally substituted with up to five fluoro; said pyrrolidinyl, piperidinyl, azepinyl in the definition of R²¹ and R²² are optionally substituted independently with up to two substituents independently selected from hydroxy, amino, hydroxy-(C₁-C₄)alkyl, (C₁-C₄)alkoxy-(C₁-C₄)alkyl, (C₁-C₄)alkyl optionally substituted with up to five fluoro and (C₁-C₄)alkoxy optionally substituted with up to five fluoro; said morpholinyl in the definition of R²¹ and R²² is optionally substituted with up to two substituents independently selected from hydroxy-(C₁-C₄)alkyl, (C₁-C₄)alkoxy-(C₁-C₄)alkyl, (C₁-C₄)alkyl optionally substituted with up to five fluoro and (C₁-C₄)alkoxy optionally substituted with up to five fluoro; said piperazinyl in the definition of R²¹ and R²² is optionally substituted independently with up to three substituents independently selected from phenyl, pyridyl, (C₁-C₄)alkoxycarbonyl and (C₁-C₄)alkyl optionally substituted with up to five fluoro; said 1,2,3,4-tetrahydroisoquinolyl and said 5,6,7,8-tetrahydropyrido[4,3-d]pyrimidyl in the definition of R²¹ and R²² are optionally substituted independently with up to three substituents independently selected from hydroxy, amino, halo, hydroxy-(C₁-C₄)alkyl, (C₁-C₄)alkoxy-(C₁-C₄)alkyl, (C₁-C₄)alkyl optionally substituted with up to five fluoro and (C₁-C₄)alkoxy optionally substituted with up to five fluoro; and said 6,7-dihydro-5H-dibenzo[c,e]azepinyl in the definition of R²¹ and R²² is optionally substituted with up to four substituents independently selected from hydroxy, amino, halo, hydroxy-(C₁-C₄)alkyl, (C₁-C₄)alkoxy-(C₁-C₄)alkyl, (C₁-C₄)alkyl optionally substituted with up to five fluoro and (C₁-C₄)alkoxy optionally substituted with up to five fluoro; said pyrimidyl, pyridyl and phenyl which are optionally substituted on said piperazine in the definition of R²¹ and R²² is optionally substituted with up to three substituents selected from hydroxy, amino, hydroxy-(C₁-C₄)alkyl, (C₁-C₄)alkoxy-(C₁-C₄)alkyl, (C₁-C₄)alkyl optionally substituted with up to five fluoro and (C₁-C₄)alkoxy optionally substituted with up to five fluoro;
Ar² is independently defined as set forth for Ar and Ar¹ above;
said Ar² is optionally independently substituted as set forth for Ar and Ar¹ above;
R²³ is CONR²⁵R²⁶ or SO₂R²⁵R²⁶, wherein R²⁵ is hydrogen (C₁-C₄)alkyl or Ar³—(C₀-C₄)alkylenyl and R²⁶ is Ar³—(C₀-C₄)alkylenyl; provided that when Ar³ is phenyl, naphthyl or biphenyl, then R²³ cannot be CONR²⁵R²⁶ where R²⁵ is hydrogen or Ar³ and R²⁶ is Ar³;
R²⁴ is hydrogen, (C₁-C₄)alkyl, (C₁-C₄)alkoxycarbonyl, (C₁-C₄)alkoxy-(C₁-C₄)alkyl, hydroxy-(C₁-C₄)alkyl or phenyl optionally independently substituted with up to three hydroxy, halo, hydroxy-(C₁-C₄)alkyl, (C₁-C₄)alkoxy-(C₁-C₄)alkyl, (C₁-C₄)alkyl or (C₁-C₄)alkoxy, wherein said (C₁-C₄)alkyl in the definition of R⁶ and said (C₁-C₄)alkoxy in the definition of R⁶ are optionally and independently substituted with up to five fluoro;
Ar³ is independently defined as set forth for Ar and Ar¹ above;
said Ar³ is optionally independently substituted as set forth for Ar and Ar¹ above;
R²⁷ is hydrogen or (C₁-C₄)alkyl;
R²⁸ and R²⁹ are each independently hydrogen, hydroxy, halo, hydroxy-(C₁-C₄)alkyl, (C₁-C₄)alkoxy-(C₁-C₄)alkyl, (C₁-C₄)alkyl optionally substituted with up to five fluoro, (C₁-C₄)alkoxy optionally substituted with up to five fluoro, phenyl, pyridyl, pyrimidyl, thienyl, furanyl, thiazolyl, oxazolyl, phenoxy, thiophenoxy, SO₂NR³⁰R³¹, CONR³⁰R³¹ or NR³⁰R³¹; said thienyl, pyrimidyl, furanyl, thiazolyl and oxazolyl in the definition of R²⁸ and R²⁹ are optionally substituted by up to two hydroxy, halo, hydroxy-(C₁-C₄)alkyl, (C₁-C₄)alkoxy-(C₁-C₄)alkyl, (C₁-C₄)alkyl optionally substituted with up to five fluoro or (C₁-C₄)alkoxy optionally substituted with up to five fluoro; said phenyl, pyridyl, phenoxy and thiophenoxy in the definition of R²⁸ and R²⁹ are optionally substituted by up to three hydroxy, halo, hydroxy-(C₁-C₄)alkyl, (C₁-C₄)alkoxy-(C₁-C₄)alkyl, (C₁-C₄)alkyl optionally substituted with up to five fluoro or (C₁-C₄)alkoxy optionally substituted with up to five fluoro;
R³⁰ and R³¹ are each independently hydrogen, (C₁-C₄)alkyl, (C₃-C₇)cycloalkyl or phenyl, said phenyl is optionally substituted with up to three hydroxy, halo, hydroxy-(C₁-C₄)alkyl, (C₁-C₄)alkoxy-(C₁-C₄)alkyl, (C₁-C₄)alkyl optionally substituted with up to five fluoro or (C₁-C₄)alkoxy optionally substituted with up to five fluoro; or
R³⁰ and R³¹ are taken together with the nitrogen to which they are attached to form indolinyl, pyrrolidinyl, piperidinyl, piperazinyl or morpholinyl; said pyrrolidinyl and piperidinyl in the definition of R³⁰ and R³¹ are optionally substituted with up to two hydroxy, amino, hydroxy-(C₁-C₄)alkyl, (C₁-C₄)alkoxy-(C₁-C₄)alkyl, (C₁-C₄)alkyl optionally substituted with up to five fluoro or (C₁-C₄)alkoxy optionally substituted with up to five fluoro; said indolinyl and piperazinyl in the definition of R³⁰ and R³¹ are optionally substituted with up to three hydroxy, amino, hydroxy-(C₁-C₄)alkyl, (C₁-C₄)alkoxy-(C₁-C₄)alkyl, (C₁-C₄)alkoxycarbonyl, (C₁-C₄)alkyl optionally substituted with up to five fluoro or (C₁-C₄)alkoxy optionally substituted with up to five fluoro; said morpholinyl in the definition of R³⁰ and R³¹ is optionally substituted with up to two substituents independently selected from hydroxy-(C₁-C₄)alkyl, (C₁-C₄)alkoxy-(C₁-C₄)alkyl, (C₁-C₄)alkyl optionally substituted with up to five fluoro and (C₁-C₄)alkoxy optionally substituted with up to five fluoro;
A is N optionally substituted with hydrogen or (C₁-C₄)alkyl and B is carbonyl; or
A is carbonyl and B is N optionally substituted with hydrogen or (C₁-C₄)alkyl;
R³² is hydrogen or (C₁-C₄)alkyl;
R³³ is phenyl, pyridyl, pyrimidyl, thiazolyl, oxazolyl, benzyl, quinolyl, isoquinolyl, phthalizinyl, quinoxanlyl, benzothiazoyl, benzoxazolyl, benzofuranyl or benzothienyl; said phenyl, pyridyl, pyrimidyl, thiazolyl, oxazolyl, benzyl, quinolyl, isoquinolyl, phthalizinyl, quinoxanlyl, benzothiazoyl, benzoxazolyl, benzofuranyl and benzothienyl in the definition of R³³ are optionally substituted with up to three phenyl, phenoxy, NR³⁴R³⁵, halo, hydroxy, hydroxy-(C₁-C₄)alkyl, (C₁-C₄)alkoxy-(C₁-C₄)alkyl, (C₁-C₄)alkyl optionally substituted with up to five fluoro or (C₁-C₄)alkoxy optionally substituted with up to five fluoro;
R³⁴ and R³⁵ are each independently hydrogen, (C₁-C₄ alkyl), phenyl or phenylsulfonyl;
said phenyl and phenylsulfonyl in the definition of R³⁴ and R³⁵ are optionally substituted with up to three halo, hydroxy, (C₁-C₄)alkyl optionally substituted with up to five fluoro or (C₁-C₄)alkoxy optionally substituted with up to five fluoro;
D is CO, CHOH or CH₂;
E is O, NH or S;
R³⁶ and R³⁷ are taken separately and are each independently hydrogen, halo, cyano, hydroxy, amino, (C₁-C₆)alkylamino, di-(C₁-C₆)alkylamino, pyrrolidino, piperidino, morpholino, (C₁-C₄)alkoxy-(C₁-C₄)alkyl, hydroxy-(C₁-C₄)alkyl, Ar⁴, (C₁-C₄)alkyl optionally substituted with up to five fluoro or (C₁-C₄)alkoxy optionally substituted with up to five fluoro;
R³⁸, R³⁹ and R⁴⁰ are each independently hydrogen or (C₁-C₄)-alkyl;
Ar⁴ is phenyl, furanyl, thienyl, pyridyl, pyrimidyl, pyrazinyl or pyridazinyl; said Ar⁴ being optionally substituted with up to three hydroxy, (C₁-C₄)alkoxy-(C₁-C₄)alkyl, halo, hydroxy-(C₁-C₄)alkyl, (C₁-C₄)alkyl optionally substituted with up to five fluoro or (C₁-C₄)alkoxy optionally substituted with up to five fluoro; or
R³⁶ and R³⁷ are taken together on adjacent carbon atoms and are —O—(CH₂)ₜ—O—;
t is 1, 2 or 3;
Y is (C₂-C₆)alkylene;
R⁴⁴, R⁴⁵ and R⁴⁶ are each independently hydrogen or (C₁-C₄)alkyl;
m and n are each independently 1, 2 or 3, provided that the sum of m and n is 2, 3 or 4;
k is 0, 1, 2, 3 or 4;
Y¹ is a covalent bond, carbonyl, sulfonyl or oxycarbonyl;
R⁴³ is (C₃-C₇)cycloalkyl, Ar⁵—(C₀-C₄)alkylenyl, NR⁴⁷R⁴⁸ or (C₁-C₆)alkyl optionally substituted with one to five fluoro; provided that when Y¹ is a covalent bond or oxycarbonyl, then R⁴³ is not NR⁴⁷R⁴⁸;
R⁴⁷ and R⁴⁸ are taken separately and are each independently selected from hydrogen, Ar⁵, (C₁-C₆)alkyl and Ar⁵—(C₀-C₄)alkylenyl; or
R⁴⁷ and R⁴⁸ are taken together with the nitrogen atom to which they are attached to form azetidinyl, pyrrolidinyl, piperidinyl, piperazinyl, morpholinyl, azepinyl, azabicyclo[3.2.2]nonanyl, azabicyclo[2.2.1]heptyl, 1,2,3,4-tetrahydroisoquinolyl, 6,7-dihydro-5H-dibenzo[c,e]azepinyl or 5,6,7,8-tetrahydropyrido[4,3-d]pyrimidyl; said azetidinyl in the definition of R⁴⁷ and R⁴⁸ are optionally substituted with one hydroxy, amino, hydroxy-(C₁-C₄)alkyl, (C₁-C₄)alkoxy-(C₁-C₄)alkyl, (C₁-C₄)alkyl optionally substituted with up to five fluoro or (C₁-C₄)alkoxy optionally substituted with up to five fluoro; said pyrrolidinyl, piperidinyl and azepinyl in the definition of R⁴⁷ and R⁴⁸ are optionally substituted with up to two hydroxy, amino, hydroxy-(C₁-C₄)alkyl, (C₁-C₄)alkoxy-(C₁-C₄)alkyl, (C₁-C₄)alkyl optionally substituted with up to five fluoro or (C₁-C₄)alkoxy optionally substituted with up to five fluoro; said morpholinyl in the definition of R⁴⁷ and R⁴⁸ is optionally substituted with up to two substituents independently selected from hydroxy-(C₁-C₄)alkyl, (C₁-C₄)alkoxy-(C₁-C₄)alkyl, (C₁-C₄)alkyl optionally substituted with up to five fluoro and (C₁-C₄)alkoxy optionally substituted with up to five fluoro; said piperazinyl, 1,2,3,4-tetrahydroisoquinolyl and 5,6,7,8-tetrahydro[4,3-d]pyrimidyl in the definition of R⁴⁷ and R⁴⁸ are optionally substituted with up to three hydroxy, amino, halo, hydroxy-(C₁-C₄)alkyl, (C₁-C₄)alkoxy-(C₁-C₄)alkyl, (C₁-C₄)alkyl optionally substituted with up to five fluoro or (C₁-C₄)alkoxy optionally substituted with up to five fluoro; and said 6,7-dihydro-5H-dibenzo[c,e]azepinyl in the definition of R⁴⁷ and R⁴⁸ are optionally substituted with up to four hydroxy, amino, halo, hydroxy-(C₁-C₄)alkyl, (C₁-C₄)alkoxy-(C₁-C₄)alkyl, (C₁-C₄)alkyl optionally substituted with up to five fluoro or (C₁-C₄)alkoxy optionally substituted with up to five fluoro;
Ar⁵ is independently defined as set forth for Ar and Ar¹ above;
Ar⁵ is optionally independently substituted as set forth for Ar and Ar¹ above;
R⁴² and R^{42a} are independently hydrogen, (C₃-C₇)cycloalkyl, Ar⁶—(C₀-C₃)alkylenyl, Ar⁶—(C₀-C₃)alkylenyl, Ar⁶—(C₂-C₄)alkenyl, Ar⁶-carbonyl or (C₁-C₆)alkyl optionally substituted with up to five fluoro;
Ar⁶ is independently defined as set forth for Ar and Ar¹ above;
Ar⁶ is optionally independently substituted as set forth for Ar and Ar¹ above; and
R⁴¹ and R^{41a} are each independently hydrogen or (C₁-C₄)alkyl.

7. A pharmaceutical composition of claim 6, wherein in said compound of Formula I, said prodrug thereof or said pharmaceutically acceptable salt of said compound or said prodrug:
R¹ is C(OH)R⁴R⁵, where R⁴ and R⁵ are each independently hydrogen or methyl;
R² is hydrogen;
R³ is 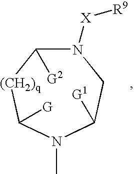
wherein said R³ is substituted by R⁶, R⁷ or R⁸;
G, G¹ and G² are taken separately and are each hydrogen and R⁶ is hydrogen or (C₁-C₄)alkyl; R⁷ and R⁸ are each independently hydrogen or (C₁-C₄)alkyl; or
G and G¹ are taken together and are (C₁-C₃)alkylene and R⁶, R⁷, R⁸ and G² are hydrogen; or
G¹ and G² are taken together and are (C₁-C₃)alkylene and R⁶, R⁷, R⁸ and G are hydrogen;
q is 0 or 1;
X is a covalent bond, oxycarbonyl,vinylenylcarbonyl, oxy(C₁-C₄)alkylenylcarbonyl, thio(C₁-C₄)alkylenylcarbonyl or vinylenylsulfonyl; said vinylenylcarbonyl and said vinylenylsulfonyl in the definition of X are optionally substituted on one or two vinylenyl carbons with (C₁-C₄)alkyl, benzyl or Ar; said oxy(C₁-C₄)alkylenylcarbonyl and said thio(C₁-C₄)alkylenylcarbonyl in the definition of X are optionally substituted with up to two (C₁-C₄)alkyl, benzyl or Ar;
R⁹ is (C₃-C₇)cycloalkyl, Ar¹—(C₀-C₄)alkylenyl or (C₁-C₆)alkyl optionally substituted with up to five fluoro;
Ar¹ is phenyl, naphthyl, pyridyl, pyrimidyl, pyrazinyl, pyridazinyl, triazinyl, quinolyl, isoquinolyl, quinazolyl, quinoxalyl, phthalazinyl, cinnolinyl, naphthyridinyl, pteridinyl, pyrazinopyrazinyl, pyrazinopyridazinyl, pyrimidopyridazinyl, pyrimidopyrimidyl, pyridopyrimidyl, pyridopyrazinyl, pyridopyridazinyl, pyrrolyl, furanyl, thienyl, imidazolyl, oxazolyl, thiazolyl, pyrazolyl, isoxazolyl, isothiazolyl, triazolyl, oxadiazolyl, thiadiazolyl, tetrazolyl, indolyl, benzofuranyl, benzothienyl, benzimidazolyl, benzoxazolyl, benzothiazolyl, indazolyl, benzisoxazolyl, benzisothiazolyl, pyrrolopyridyl, furopyridyl, thienopyridyl, imidazolopyridyl, oxazolopyridyl, thiazolopyridyl, pyrazolopyridyl, isoxazolopyridyl, isothiazolopyridyl, pyrrolopyrimidyl, furopyrimidyl, thienopyrimidyl, imidazolopyrimidyl, oxazolopyrimidyl, thiazolopyrimidyl, pyrazolopyrimidyl, isoxazolopyrimidyl, isothiazolopyrimidyl, pyrrolopyrazinyl, furopyrazinyl, thienopyrazinyl, imidazolopyrazinyl, oxazolopyrazinyl, thiazolopyrazinyl, pyrazolopyrazinyl, isoxazolopyrazinyl, isothiazolopyrazinyl, pyrrolopyridazinyl, furopyridazinyl, thienopyridazinyl, imidazolopyridazinyl, oxazolopyridazinyl, thiazolopyridazinyl, pyrazolopyridazinyl, isoxazolopyridazinyl or isothiazolopyridazinyl; and
said Ar¹ is optionally substituted as set forth in claim 6.

8. A pharmaceutical composition of claim 7, wherein in said compound of Formula I, said prodrug thereof or said pharmaceutically acceptable salt of said compound or said prodrug:
X is a covalent bond, oxycarbonyl or vinylenylcarbonyl optionally substituted on one or two vinylenyl carbons with (C₁-C₄)alkyl, benzyl or Ar;
R⁹ is Ar¹—(C₀-C₄)alkylenyl;
Ar¹ is phenyl, naphthyl, pyridyl, pyrimidyl, pyrazinyl, triazinyl, quinolyl, isoquinolyl, quinazolyl, quinoxalyl, furanyl, thienyl, indolyl, benzofuranyl, benzothienyl, benzoxazolyl, benzothiazolyl, furopyridyl, oxazolopyridyl, thiazolopyridyl, thienopyridyl, furopyrimidyl, thienopyrimidyl, oxazolopyrimidyl or thiazolopyrimidyl; and
said Ar¹ is optionally substituted as set forth in claim 7.

9. A pharmaceutical composition of claim 8 wherein in said compound of Formula I, said prodrug thereof or said pharmaceutically acceptable salt of said compound or said prodrug:
R² is hydrogen;
R⁴ is hydrogen or methyl;
R⁵ is methyl;
G, G¹ and G² are hydrogen;
R⁶ and R⁷ are each independently hydrogen or methyl;
R⁸ is hydrogen.

10. A pharmaceutical composition of claim 9 wherein said compound of Formula I is selected from
1R-(4-{1′-[2-(1R-hydroxy-ethyl)-pyrimidin-4-yl]-[4,4′]bipiperidinyl-1-yl}-pyrimidin-2-yl)-ethanol;
furo[3,2-c]pyridin-2-yl-{4-[2-(1R-hydroxy-ethyl)-pyrimidin-4-yl]-3R,5S-dimethyl-piperazin-1-yl}-methanone;
(4-chloro-furo[3,2-c]pyridin-2-yl)-{4-[2-(1R-hydroxy-ethyl)-pyrimidin-4-yl]-3R,5S-dimethyl-piperazin-1-yl}-methanone;
{4-[2-(1R-hydroxy-ethyl)-pyrimidin-4-yl]-3R,5S-dimethyl-piperazin-1-yl}-(4-pyrrolidin-1-yl-furo[3,2-c]pyridin-2-yl)-methanone;
{4-[2-(1R-hydroxy-ethyl)-pyrimidin-4-yl]-3R,5S-dimethyl-piperazin-1-yl}-(4-morpholin-4-yl-furo[3,2-c]pyridin-2-yl)-methanone;
{4-[2-(1R-hydroxy-ethyl)-pyrimidin-4-yl]-3R,5S-dimethyl-piperazin-1-yl}-imidazo[1,2-a]pyridin-2-yl-methanone;
furo[3,2-c]pyridin-2-yl-{4-[2-(1R-hydroxy-ethyl)-pyrimidin-4-yl]-2R,6S-dimethyl-piperazin-1-yl}-methanone;
4-[2-(1R-hydroxy-ethyl)-pyrimidin-4-yl]-2R,6S-dimethyl-piperazine-1-carboxylic acid pyridin-3-yl ester;
4-[2-(1R-hydroxy-ethyl)-pyrimidin-4-yl]-2R,6S-dimethyl-piperazine-1-carboxylic acid 2-methyl-pyridin-3-yl ester;
4-[2-(1R-hydroxy-ethyl)-pyrimidin-4-yl]-2R,6S-dimethyl-piperazine-1-carboxylic acid 5-chloro-pyridin-3-yl ester;
4-[2-(1R-hydroxy-ethyl)-pyrimidin-4-yl]-2R,6S-dimethyl-piperazine-1-carboxylic acid 6-methyl-pyridin-3-yl ester;
(E)-1-{4-[2-(1R-hydroxy-ethyl)-pyrimidin-4-yl]-2R,6S-dimethyl-piperazin-1-yl}-3-thiophen-2-yl-propenone;
1R-{4-[4-(4,6-dimethyl-pyrimidin-2-yl)-3R,5S-dimethyl-piperazin-1-yl]-pyrimidin-2-yl}-ethanol;
1R-{4-[4-(4-methoxymethyl-6-methyl-pyrimidin-2-yl)-3R,5S-dimethyl-piperazin-1-yl]-pyrimidin-2-yl}-ethanol;
1R-{4-[4-(4-hydroxymethyl-6-methyl-pyrimidin-2-yl)-3R,5S-dimethyl-piperazin-1-yl]-pyrimidin-2-yl}-ethanol;
1R-(4-{3R,5S-dimethyl-4-[2-(4-methyl-piperazin-1-yl)-pyrimidin-4-yl]-piperazin-1-yl}-pyrimidin-2-yl)-ethanol;
1R-(4-{4-[2-(4-ethyl-piperazin-1-yl)-pyrimidin-4-yl]-3R,5S-dimethyl-piperazin-1-yl}-pyrimidin-2-yl)-ethanol;
1R-(4-{3R,5S-dimethyl-4-[2-(4-methyl-imidazol-1-yl)-pyrimidin-4-yl]-piperazin-1-yl}-pyrimidin-2-yl)-ethanol;
1R-(4-{3R,5S-dimethyl-4-[2-(2-methyl-imidazol-1-yl)-pyrimidin-4-yl]-piperazin-1-yl}-pyrimidin-2-yl)-ethanol;
1R-(4-{4-[2-(2,4-dimethyl-imidazol-1-yl)-pyrimidin-4-yl]-3R,5S-dimethyl-piperazin-1-yl-}-pyrimidin-2-yl)-ethanol;
1R-(4-{4-[2-(4-isopropyl-piperazin-1-yl)-pyrimidin-4-yl]-3R,5S-dimethyl-piperazin-1-yl}-pyrimidin-2-yl)-ethanol;
1R-(4-{3R,5S-dimethyl-4-[4-methyl-6-(4-methyl-piperazin-1-yl)-[1,3,5]triazin-2-yl]-piperazin-1-yl}-pyrimidin-2-yl)-ethanol;
1R-{4-[4-(4-methoxy-6-methyl-[1,3,5]triazin-2-yl)-3R,5S-dimethyl-piperazin-1-yl]-pyrimidin-2-yl}-ethanol;
1R-{4-[4-(4,6-dimethoxy-[1,3,5]triazin-2-yl)-3R,5S-dimethyl-piperazin-1-yl]-pyrimidin-2-yl}-ethanol;
1R-{4-[4-(4-ethoxy-6-methyl-[1,3,5]triazin-2-yl)-3R,5S-dimethyl-piperazin-1-yl]-pyrimidin-2-yl}-ethanol;
1R-{4-[4-(4-isopropoxy-6-methyl-[1,3,5]triazin-2-yl)-3R,5S-dimethyl-piperazin-1-yl]-pyrimidin-2-yl}-ethanol;
1R-{4-[3R,5S-dimethyl-4-(4-phenyl-[1,3,5]triazin-2-yl)-piperazin-1-yl]-pyrimidin-2-yl}-ethanol;
1R-{4-[4-(4-hydroxymethyl-6-methoxy-[1,3,5]triazin-2-yl)-3R,5S-dimethyl-piperazin-1-yl]-pyrimidin-2-yl}-ethanol;
1R-{4-[4-(4-isopropoxy-6-methoxy-[1,3,5]triazin-2-yl)-3R,5S-dimethyl-piperazin-1-yl]-pyrimidin-2-yl}-ethanol;
1R-{4-[4-(4-isopropyl-[1,3,5]triazin-2-yl)-3R,5S-dimethyl-piperazin-1-yl]-pyrimidin-2-yl}-ethanol;
1R-{4-[4-(4-ethyl-6-methoxy-[1,3,5]triazin-2-yl)-3R,5S-dimethyl-piperazin-1-yl]-pyrimidin-2-yl}-ethanol;
1R-{4-[4-(4,6-dimethyl-pyrimidin-2-yl)-2R,6S-dimethyl-piperazin-1-yl]-pyrimidin-2-yl}-ethanol;
1R-{4-[4-(4-hydroxymethyl-6-methyl-pyrimidin-2-yl)-2R,6S-dimethyl-piperazin-1-yl]-pyrimidin-2-yl}-ethanol;
1R-{4-[2R,6S-dimethyl-4-(4-[1,2,4]triazol-1-yl-pyrimidin-2-yl)-piperazin-1-yl]-pyrimidin-2-yl-}-ethanol;
1R-{4-[4-(2,6-dimethyl-pyrimidin-4-yl)-2R,6S-dimethyl-piperazin-1-yl]-pyrimidin-2-yl}-ethanol;
1R-(4-{4-[2-(1R-hydroxy-ethyl)-pyrimidin-4-yl]-2R,6S-dimethyl-piperazin-1-yl}-pyrimidin-2-yl)-ethanol;
1R-{4-[4-(2-hydroxymethyl-6-methyl-pyrimidin-4-yl)-2R,6S-dimethyl-piperazin-1-yl]-pyrimidin-2-yl}-ethanol;
1R-(4-{4-[2-(1S-hydroxy-ethyl)-pyrimidin-4-yl]-2R,6S-dimethyl-piperazin-1-yl}-pyrimidin-2-yl)-ethanol;
1S-(4-{4-[2-(1R-hydroxy-ethyl)-pyrimidin-4-yl]-2R,6S-dimethyl-piperazin-1-yl}-pyrimidin-2-yl)-ethanol;
1-{4-[4-(2-acetyl-pyrimidin-4-yl)-2R,6S-dimethyl-piperazin-1-yl]-pyrimidin-2-yl}-ethanone;
1RS-(4-{4-[2-(1RS-hydroxy-ethyl)-pyrimidin-4-yl]-2R,6S-dimethyl-piperazin-1-yl}-pyrimidin-2-yl)-ethanol;
(4-{4-[2-(1R-hydroxy-ethyl)-pyrimidin-4-yl]-3R,5S-dimethyl-piperazin-1-yl}-pyrimidin-2-yl)-ethanone;
1R-{4-[2R,6S-dimethyl-4-(2-morpholin-4-yl-pyrimidin-4-yl)-piperazin-1-yl]-pyrimidin-2-yl}-ethanol;
1R-(4-{2R,6S-dimethyl-4-[2-(4-methyl-piperazin-1-yl)-pyrimidin-4-yl]-piperazin-1-yl}-pyrimidin-2-yl)-ethanol;
1R-{4-[2R,6S-dimethyl-4-(2-[1,2,4]triazol-1-yl-pyrimidin-4-yl)-piperazin-1-yl]-pyrimidin-2-yl}-ethanol;
1R-(4-{4-[2-(1R-hydroxy-ethyl)-pyrimidin-4-yl]-2R,6R-dimethyl-piperazin-1-yl}-pyrimidin-2-yl)-ethanol;
1R-(4-{4-[2-(4-ethyl-piperazin-1-yl)-pyrimidin-4-yl]-2R,6S-dimethyl-piperazin-1-yl}-pyrimidin-2-yl)-ethanol;
1R-(4-{2R,6S-dimethyl-4-[2-(4-methyl-imidazol-1-yl)-pyrimidin-4-yl]-piperazin-1-yl}-pyrimidin-2-yl)-ethanol;
1R-(4-{4-[2-(2,4-dimethyl-imidazol-1-yl)-pyrimidin-4-yl]-2R,6S-dimethyl-piperazin-1-yl}-pyrimidin-2-yl)-ethanol;
1R-{4-[2R,6S-dimethyl-4-(4-morpholin-4-yl-[1,3,5]triazin-2-yl)-piperazin-1-yl]-pyrimidin-2-yl}-ethanol;
1R-{4-[4-(4-methoxy-6-methyl-[1,3,5]triazin-2-yl)-2R,6S-dimethyl-piperazin-1-yl]-pyrimidin-2-yl}-ethanol;
1R-{4-[4-(4,6-dimethoxy-[1,3,5]triazin-2-yl)-2R,6S-dimethyl-piperazin-1-yl]-pyrimidin-2-yl}-ethanol;
1R-{4-[2R,6S-dimethyl-4-(4-phenyl-[1,3,5]triazin-2-yl)-piperazin-1-yl]-pyrimidin-2-yl}-ethanol;
1R-{4-[4-(4-hydroxymethyl-6-methyl-pyrimidin-2-yl)-3S-methyl-piperazin-1-yl]-pyrimidin-2-yl}-ethanol;
1R-{4-[4-(2-hydroxymethyl-pyrimidin-4-yl)-3S-methyl-piperazin-1-yl]-pyrimidin-2-yl}-ethanol;
1R-{4-[4-(2-hydroxymethyl-6-methyl-pyrimidin-4-yl)-3S-methyl-piperazin-1-yl]-pyrimidin-2-yl}-ethanol;
1R-[4-(3S-methyl-4-oxazolo[5,4-b]pyridin-2-yl-piperazin-1-yl)-pyrimidin-2-yl]-ethanol;
1R-[4-(3S-methyl-4-oxazolo[4,5-b]pyridin-2-yl-piperazin-1-yl)-pyrimidin-2-yl]-ethanol;
1R-[4-(3S-methyl-4-quinoxalin-2-yl-piperazin-1-yl)-pyrimidin-2-yl]-ethanol;
1R-{4-[4-(4,6-dimethyl-[1,3,5]triazin-2-yl)-3R,5S-dimethyl-piperazin-1-yl]-pyrimidin-2-yl}-ethanol;
1R-{4-[3R,5S-dimethyl-4-(4-methyl-6-phenyl-[1,3,5]triazin-2-yl)-piperazin-1-yl]pyrimidin-2-yl}-ethanol;
1R-{4-[4-(4-cyclopropyl-[1,3,5]triazin-2-yl)-3R,5S-dimethyl-piperazin-1-yl]-pyrimidin-2-yl}-ethanol;
1R-{4-[4-(4-cyclopropyl-[1,3,5]triazin-2-yl)-2R, 6S-dimethyl-piperazin-1-yl]-pyrimidin-2-yl}-ethanol;
1R-{4-[4-(4,6-dimethyl-[1,3,5]triazin-2-yl)-2R,6S-dimethyl-piperazin-1-yl]-pyrimidin-2-yl}-ethanol;
1R-{4-[4-(4-hydroxymethyl-6-phenyl-[1,3,5]triazin-2-yl)-2R ,6S-dimethyl-piperazin-1-yl]-pyrimidin-2-yl}-ethanol;
1R-{4-[4-(4-methoxy-6-methoxymethyl-[1,3,5]triazin-2-yl)-2R,6S-dimethyl-piperazin-1-yl]-pyrimidin-2-yl}-ethanol;
1R-{4-[2R ,6S-dimethyl-4-(4-methyl-[1,3,5]triazin-2-yl)-piperazin-1-yl]-pyrimidin-2-yl-ethanol;
1-{4-[4-(2-acetyl-pyrimidin-4-yl)-2R*,6S*-dimethyl-piperazin-1-yl]-pyrimidin-2-yl}-ethanone;
1-(-4-{4-[2-(1R-hydroxy-ethyl)-pyrimidin-4-yl]-2R,6S-dimethyl-piperazin-1-yl}-pyrimidin-2-yl)-ethanone;
1R-{4-[4-(4-methoxymethyl-6-phenyl-[1,3,5]-triazin-2-yl)-2R,6S-dimethyl-piperazin-1-yl]-pyrimidin-2-yl}-ethanol;
(4-{4-[2-(1R-hydroxy-ethyl)-pyrimidin-4-yl]-2R,5S-dimethyl-piperazin-1-yl}-pyrimidin-2-yl)-ethanol; 1S-(4-{4-[2-(1R-hydroxy-ethyl)-pyrimidin-4-yl]-2R,6S-dimethyl-piperazin-1-yl}-pyrimidin-2-yl)-ethanol; prodrugs thereof and pharmaceutically acceptable salts of said compounds and said prodrugs.

11. A pharmaceutical composition of claim 10 wherein said compound of
Formula I is selected from 1R-{4-[4-(2,6-dimethyl-pyrimidin-4-yl)-2R,6S-dimethyl-piperazin-1-yl]-pyrimidin-2-yl}-ethanol; 1R-(4-{4-[2-(1R-hydroxy-ethyl)-pyrimidin-4-yl]-2R,6S-dimethyl-piperazin-1-yl}-pyrimidin-2-yl)-ethanol; 1R-{4-[4-(2-hydroxymethyl-6-methyl-pyrimidin-4-yl)-2R, 6S-dimethyl-piperazin-1-yl]-pyrimidin-2-yl}-ethanol; 1R-(4-{4-[2-(1-S-hydroxy-ethyl)-pyrimidin-4-yl]-2R,6S-dimethyl-piperazin-1-yl}-pyrimidin-2-yl)-ethanol; 1S-(4-{4-[2-(1R-hydroxy-ethyl)-pyrimidin-4-yl]-2R,6S-dimethyl-piperazin-1-yl}-pyrimidin-2-yl)-ethanol; 1-{4-[4-(2-acetyl-pyrimidin-4-yl)-2R,6S-dimethyl-piperazin-1-yl]-pyrimidin-2-yl}-ethanone; 1RS-(4-{4-[2-(1RS-hydroxy-ethyl)-pyrimidin-4-yl]-2R,6S-dimethyl-piperazin-1-yl}-pyrimidin-2-yl)-ethanol; (4-{4-[2-(1R-hydroxy-ethyl)-pyrimidin-4-yl]-3R,5S-dimethyl-piperazin-1-yl}-pyrimidin-2-yl)-ethanone; 1R-{4-[2R,6S-dimethyl-4-(2-morpholin-4-yl-pyrimidin-4-yl)-piperazin-1-yl]-pyrimidin-2-yl}-ethanol; 1R-(4-{2R,6S-dimethyl-4-[2-(4-methyl-piperazin-1-yl)-pyrimidin-4-yl]-piperazin-1-yl}-pyrimidin-2-yl)-ethanol; 1R-{4-[2R,6S-dimethyl-4-(2-[1,2,4]triazol-1-yl-pyrimidin-4-yl)-piperazin-1-yl]-pyrimidin-2-yl}-ethanol; 1R-(4-{4-[2-(1R-hydroxy-ethyl)-pyrimidin-4-yl]-2R,6R -dimethyl-piperazin-1-yl}-pyrimidin-2-yl)-ethanol; 1R-(4-{4-[2-(4-ethyl-piperazin-1-yl)-pyrimidin-4-yl]-2R,6S-dimethyl-piperazin-1-yl}-pyrimidin-2-yl)-ethanol; 1R-(4-{2R,6S-dimethyl-4-[2-(4-methyl-imidazol-1-yl)-pyrimidin-4-yl]-piperazin-1-yl}-pyrimidin-2-yl)-ethanol; 1R-{4-[4-(4-cyclopropyl-[1,3,5]triazin-2-yl)-2R,6S-dimethyl-piperazin-1-yl]-pyrimidin-2-yl}-ethanol; 1R-{4-[4-(4,6-dimethyl-[1,3,5]triazin-2-yl)-2R,6S-dimethyl-piperazin-1-yl]-pyrimidin-2-yl}-ethanol; 1R-{4-[4-(4-hydroxymethyl-6-phenyl-[1,3,5]triazin-2-yl)-2R,6S-dimethyl-piperazin-1-yl]-pyrimidin-2-yl}-ethanol; 1R-{4-[4-(4-methoxy-6-methoxymethyl-[1,3,5]triazin-2-yl)-2R,6S-dimethyl-piperazin-1-yl]-pyrimidin-2-yl}-ethanol; 1R-{4-[2R,6S-dimethyl-4-(4-methyl-[1,3,5]triazin-2-yl)-piperazin-1-yl]-pyrimidin-2-yl-ethanol; and 1R-(4-{4-[2-(2,4-dimethyl-imidazol-1-yl)-pyrimidin-4-yl]-2R,6S-dimethyl-piperazin-1-yl}-pyrimidin-2-yl)-ethanol; a prodrug thereof and pharmaceutically acceptable salts of said compound or said prodrug.

12. A pharmaceutical composition of claim 11 wherein said statin is atorvastatin, simvastatin, pravastatin, rivastatin, mevastatin, fluindostatin, velostatin, fluvastatin, dalvastatin, dihydrocompactin, compactin or lovastatin; a prodrug thereof or a pharmaceutically acceptable salt of said statin or said prodrug.

13. A pharmaceutical composition of claim 12 wherein said statin is atorvastatin, rivastatin, simvastatin, pravastatin, mevastatin, lovastatin a prodrug thereof or a pharmaceutically acceptable salt of said statin or said prodrug.

14. A pharmaceutical composition of claim 13 wherein said statin is atorvastatin or atorvastatin calcium.

15. A kit for achieving a therapeutic effect in a mammal comprising:
a. an amount of a statin, a prodrug thereof or a pharmaceutically acceptable salt of said statin or said prodrug and a pharmaceutically acceptable vehicle, carrier or diluent in a first unit dosage form;
b. an amount of a sorbitol dehydrogenase inhibitor, a prodrug thereof or a pharmaceutically acceptable salt of said sorbitol dehydrogenase inhibitor or said produrg and a pharmaceutically acceptable vehicle, carrier or diluent in a second unit dosage form; and
c. a container.

16. A method for treating a mammal in need of therapeutic treatment comprising administering to said mammal
(a) an amount of a first compound, said first compound being a statin, a prodrug thereof or a pharmaceutically acceptable salt of said statin or said prodrug; and
(b) an amount of a second compound, said second compound being a sorbitol dehydrogenase inhibitor, a prodrug thereof or a pharmaceutically acceptable salt of said sorbitol dehydrogenase inhibitor or said prodrug; wherein said first compound and said second compound are each optionally and independently administered together with a pharmaceutically acceptable vehicle, carrier or diluent.

17. A method of claim 16 wherein said statin is atorvastatin or atorvastatin calcium.

18. A method of claim 16 wherein said treatment comprises treating a diabetic complication in a mammal.

19. A method of claim 18 wherein said diabetic complication is selected from diabetic neuropathy, diabetic nephropathy, diabetic cardiomyopathy, diabetic retinopathy, diabetic microangiopathy, diabetic macroangiopathy, cataracts and foot ulcers.

20. A method of claim 16 wherein said treatment comprises treating atherosclerosis in a mammal.
